(19) United States
(12) Patent Application Publication
(10) Pub. No.: US 2026/0355024 A1
(43) Pub. Date: Nov. 12, 2026

(54) Multi-Layer Absorbent Substrate and Absorbent Articles Incorporating Same

(71) Applicant: Kimberly-Clark Worldwide, Inc., Neenah, WI (US)

(72) Inventors: Stephen M. Lindsay, Appleton, WI (US); Stephen A. Marrano, Oshkosh, WI (US); Greg J. DeGrave, Combined Locks, WI (US); Kenneth R. Schueler, Jr., Appleton, WI (US); Uyen T. Lam, Menasha, WI (US); Francis P. Abuto, Johns Creek, GA (US); Jian Qin, Appleton, WI (US); Charles W. Colman, Marietta, GA (US)

(21) Appl. No.: 19/135,124

(22) PCT Filed: Dec. 8, 2023
(86) PCT No.: PCT/US2023/083109

§ 371 Date: Jun. 3, 2025

Related U.S. Application Data
(60) Provisional application No. 63/431,409, filed on Dec. 9, 2022.

(51) Int. Cl. A61F 13/539; A61F 13/15; A61F 13/49; A61F 13/53; A61L 15/22; A61L 15/60

(52) U.S. Cl. CPC A61F 13/539; A61F 13/15203; A61F 13/49; A61L 15/225; A61L 15/60; A61F 2013/15292; A61F 2013/15471; A61F 2013/530021; A61F 2013/53024

(57) ABSTRACT

Multi-layer substrates are disclosed that have an excellent combination of tensile strength and water handling characteristics. The substrates can be produced according to a foam-forming process. Binder fibers are strategically located in outer layers while being minimized in an inner layer in order to produce an absorbent structure that can receive significant amounts of liquids and expand without restriction.

### RELATED APPLICATIONS

The present application is based upon and claims priority to U.S. Provisional Patent Application Ser. No. 63/431,409, having a filing date of Dec. 9, 2022, which is incorporated herein by reference in its entirety.

### BACKGROUND

Many different types of nonwoven materials exist that are designed to have different functions. In many embodiments, the nonwoven materials are designed to have liquid handling properties. These nonwoven materials can be used in absorbent articles to absorb fluids.

Absorbent articles, also referred to as personal care products, such as diapers, diaper pants, training pants, adult incontinence products, and feminine care products can include a variety of substrates. For example, absorbent articles can include an absorbent structure, nonwoven materials, and films. These layers are positioned relative to each other so that fluids coming into contact with the absorbent article are quickly drawn into the absorbent structure and contained in order to give the wearer a dry feel.

Absorbent structures can contain a superabsorbent material. Superabsorbent materials can be configured in the form of particles including fibers and are commonly utilized in substrates for increased absorbent capacity.

One problem in designing absorbent articles is the ability to produce an absorbent structure containing superabsorbent materials that is soft, flexible, strong, thin, and has good absorbent properties. Making the absorbent structure solely from superabsorbent materials, for instance, does not possess strength or a consolidated substrate that can be easily handled. Thus, in the past, various different fibers have been incorporated into absorbent structures including binder fibers. Binder fibers can increase the strength and integrity of the absorbent structure but can impede absorbency performance. Binder fibers, for instance, can have hydrophobic properties and can produce a bound network that resists or slows volume expansion as the absorbent structure swells when contacted with fluids reducing the available void volume of the absorbent structure. Thus, a need currently exists for an improved absorbent structure that has a balance of strength and absorbency characteristics.

### SUMMARY

The present disclosure is generally directed to a multi-layer liquid absorbent substrate that has excellent absorbency properties in combination with excellent physical strength properties. The present disclosure is also directed to all different types of absorbent articles incorporating the liquid absorbent substrate. The substrates can be made through a foam forming process.

In one aspect, for instance, the present disclosure is directed to a multi-layer substrate comprising a first layer containing binder fibers. The substrate further includes a second layer comprising a superabsorbent material. In accordance with the present disclosure, the multi-layer substrate has a tensile strength in one direction of greater than about 1,000 gf/3 in and displays a 30 second AUL test result of greater than about 10 g/g. For example, the multi-layer substrate can have a tensile strength in at least one direction of greater than about 1,500 gf/3 in, such as greater than about 2,000 gf/3 in, such as greater than about 3,000 gf/3 in, such as greater than about 3,500 gf/3 in, such as greater than about 4,000 gf/3 in, such as greater than about 4,500 gf/3 in, such as greater than about 5,000 gf/3 in, such as greater than about 5,500 gf/3 in, such as greater than about 6,000 gf/3 in, such as greater than about 6,500 gf/3 in, such as greater than about 7,000 gf/3 in, such as greater than about 7,500 gf/3 in, such as greater than about 8,000 gf/3 in, such as greater than about 8,500 gf/3 in, such as greater than about 9,000 gf/3 in, such as greater than about 9,500 gf/3 in, such as greater than about 10,000 gf/3 in, and generally less than about 20,000 gf/3 in. The normalized tensile strength can be greater than about 333 gf/in, such as greater than about 500 gf/in, such as greater than about 700 gf/in, such as greater than about 1,000 gf/in, such as greater than about 1,200 gf/in, such as greater than about 1,300 gf/in, such as greater than about 1,500 gf/in, such as greater than about 2,000 gf/in, such as greater than about 2,500 gf/in, such as greater than about 3,000 gf/in, and generally less than about 10,000 gf/in, such as less than about 7,000 gf/in.

The multi-layer substrate can display a 30 second AUL test result of greater than about 11 g/g, such as greater than about 12 g/g, such as greater than about 13 g/g, such as greater than about 14 g/g, such as greater than about 15 g/g, such as greater than about 16 g/g, such as greater than about 17 g/g, such as greater than about 18 g/g, and less than about 30 g/g. The multi-layer substrate can also display an area-normalized saturation capacity of greater than about 0.25 g/cm², such as greater than about 0.5 g/cm², than about 0.8 g/cm², such as greater than about 1.0 g, such as greater than about 1.2 g, such as greater than about 1.5 g/cm², and less than about 2.5 g/cm².

As described above, the second layer contains the superabsorbent material. In one aspect, the second layer contains little or no binder fibers. For instance, binder fibers can be present in the second layer in an amount less than about 20% by weight, such as in an amount less than about 8% by weight, such as in an amount less than about 5% by weight, such as in an amount less than about 4% by weight, such as in an amount less than about 3% by weight, such as in an amount less than about 2% by weight, such as in an amount less than about 1% by weight. In one aspect, the amount of binder fibers contained in the second layer is less than about 5% by weight based upon the total amount of fibers contained in the second layer.

The second layer can contain the superabsorbent material in an amount greater than about 20% by weight, such as in an amount greater than about 30% by weight, such as in an amount greater than about 40% by weight, such as in an amount greater than about 50% by weight, such as in an amount greater than about 60% by weight, such as in an amount greater than about 70% by weight, such as in an amount greater than about 80% by weight, such as in an amount greater than about 90% by weight, such as in an amount greater than about 95% by weight. The second layer can contain the superabsorbent material in an amount less than about 100% by weight, such as in an amount less than about 90% by weight, such as in an amount less than about 80% by weight, such as in an amount less than about 70% by weight. The second layer can also contain various different types of fibers. For instance, the second layer can contain pulp fibers, such as crosslinked pulp fibers, non-crosslinked pulp fibers, or mixtures thereof, polymer synthetic fibers, such as polyester fibers, or mixtures thereof. The second layer can have a basis weight of greater than about 200 gsm, such as greater than about 250 gsm, such as greater than about 300 gsm, such as greater than about 350 gsm, and less than about 800 gsm, such as less than about 600 gsm.

The first layer of the multi-layer substrate, on the other hand, can contain relatively great amounts of binder fibers in comparison to the second layer. For instance, the first layer can contain binder fibers in an amount greater than about 20% by weight, such as in an amount greater than about 30% by weight, such as in an amount greater than about 40% by weight, such as in an amount greater than about 50% by weight, and in an amount up to 100% by weight, such as in an amount less than about 80% by weight, such as in an amount less than about 70% by weight. The binder fibers can comprise bicomponent fibers having a core polymer and a sheath polymer. The sheath polymer can have a lower melting temperature that causes the binder fibers to bond to other binder fibers or other materials contained in the substrate when heated. In one aspect, the binder fibers include a core polymer made from a polyester polymer and a sheath polymer made from a polyethylene polymer. In addition to binder fibers, the first layer can also contain pulp fibers including crosslinked pulp fibers, synthetic polymer fibers such as polyester fibers, and mixtures thereof. In one aspect, the first layer contains superabsorbent material in an amount less than about 2% by weight and/or can be free of superabsorbent material.

The first layer can have a basis weight of from about 10 gsm to about 100 gsm, including all increments of 1 gsm therebetween. In one aspect, the first layer has a basis weight of greater than about 35 gsm, such as greater than about 40 gsm, such as greater than about 45 gsm, such as greater than about 50 gsm, and less than about 100 gsm, such as less than about 90 gsm, such as ess than about 80 gsm. In this embodiment, the first layer can contain binder fibers in an amount from about 20% to about 80% by weight and can be combined with synthetic polymer fibers.

In another aspect, the first layer can have a basis weight of less than about 35 gsm, such as less than about 30 gsm, such as less than about 25 gsm, such as less than about 20 gsm, and greater than about 10 gsm, such as greater than about 15 gsm, such as greater than about 20 gsm, In this embodiment, the first layer can contain binder fibers in an amount from about 10% to about 80% by weight. The binder fibers can be combined with pulp fibers, including crosslinked pulp fibers. The first layer can optionally contain polymer synthetic fibers.

In one embodiment, the multi-layer substrate can optionally include a third layer. The second absorbent layer can be positioned between the first layer and the third layer. In one aspect, the first layer can form a top exterior layer that is configured to face a wearer when incorporated into an absorbent article. The third layer, on the other hand, can form the bottom exterior layer. In one aspect, the third layer can comprise any suitable layer capable of preventing superabsorbent material from escaping the multi-layer substrate from the second layer. The third layer, for instance, can have a basis weight of from about 5 gsm to about 50 gsm, including all increments of 1 gsm therebetween. For example, the third layer can have a basis weight of greater than about 10 gsm, such as greater than about 15 gsm, and less than about 50 gsm, such as less than about 40 gsm, such as less than about 30 gsm, such as less than about 25 gsm, such as less than about 20 gsm. The third layer can contain binder fibers alone or in combination with pulp fibers and/or synthetic polymer fibers. The pulp fibers can optionally comprise crosslinked pulp fibers. The binder fibers, in one aspect, can have a size of less than about 6 denier, such as less than about 4 denier, such as less than about 2.5 denier, such as less than about 2 denier, such as less than about 1.5 denier, and greater than about 0.3 denier.

The present disclosure is also directed to absorbent articles incorporating the multi-layer substrate as described above. The absorbent article, for instance, can include a fluid permeable liner, an outer cover, and an absorbent core comprising the multi-layer substrate of the present disclosure positioned between the liner and the outer cover. In one aspect, the absorbent article can comprise a diaper or child's pant, including a training pant. Alternatively, the absorbent article can comprise an adult incontinence product.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1A is a side plan view of an exemplary multi-layer absorbent material including three layers according to one embodiment of the present disclosure;

FIG. 1B is a side plan view of an exemplary multi-layer absorbent material including two layers according to another embodiment of the present disclosure;

FIG. 2 is a process schematic of an exemplary apparatus and associated method for forming a multi-layer absorbent material;

FIG. 3 is a detailed view of the headbox, headbox inputs, and resultant slurry from the headbox of FIG. 2;

FIG. 4 is a side plan view of an alternative apparatus and associated method that can be used for forming a multi-layer absorbent material;

FIG. 5 is a perspective view of exemplary equipment for performing the Absorbency Under Load (AUL) Test described herein; and

FIG. 6 is a perspective view of the plastic platen weight that is inserted into the AUL cylinder shown in FIG. 5.

FIG. 7 is a perspective view of exemplary equipment for performing the Fluid Intake Under Pressure (FIUP) Test described herein with the cover being opened.

FIG. 8 is a perspective view of the exemplary equipment of FIG. 7 with the cover being closed.

Repeat use of reference characters in the present specification and drawings is intended to represent the same or analogous features or elements of the present invention.

### DEFINITIONS

As used herein, the term “foam formed product” means a product formed from a suspension including a mixture of a solid, a liquid, and dispersed gas bubbles.

As used herein, the term “foam forming process” means a process for manufacturing a product involving a suspension including a mixture of a solid, a liquid, and dispersed gas bubbles.

As used herein, the term “foaming fluid” means any one or more known fluids compatible with the other components in the foam forming process. Suitable foaming fluids include, but are not limited to, water.

As used herein, the term “foam half life” means the time elapsed until the half of the initial frothed foam mass reverts to liquid water.

As used herein, the term “layer” refers to a structure that provides an area of a substrate in a height direction of the substrate that is comprised of similar components and structure.

As used herein, the term “nonwoven web” means a web having a structure of individual fibers or threads which are interlaid, but not in an identifiable manner as in a knitted web.

As used herein, unless expressly indicated otherwise, when used in relation to material compositions the terms “percent”, “%”, “weight percent”, or “percent by weight” each refer to the quantity by weight of a component as a percentage of the total except as whether expressly noted otherwise.

The term “absorbent article” refers herein to an article intended and/or adapted to be placed against or in proximity to the body (i.e., contiguous with the body) of the wearer to absorb and contain various liquid, solid, and semi-solid exudates discharged from the body. Examples include, but are not limited to, diapers, diaper pants, training pants, youth pants, swim pants, feminine hygiene products, including, but not limited to, menstrual pads or pants, incontinence products, medical garments, surgical pads and bandages, and so forth.

The term “superabsorbent material” as used herein refers to water-swellable, water-insoluble organic or inorganic materials including superabsorbent polymers and superabsorbent polymer compositions capable, under the most favorable conditions, of absorbing at least about 10 times their weight, or at least about 15 times their weight, or at least about 25 times their weight in an aqueous solution containing 0.9 weight percent sodium chloride.

The term “machine direction” as used herein refers to the direction of travel of the forming surface onto which fibers are deposited during formation of a nonwoven web.

The term “cross-machine direction” as used herein refers to the direction which is perpendicular to both the machine direction and the height direction defined above.

The term “pulp” as used herein refers to fibers from natural sources such as woody and non-woody plants. Woody plants include, for example, deciduous and coniferous trees. Non-woody plants include, for example, cotton, flax, esparto grass, milkweed, straw, jute, hemp, and bagasse. Pulp fibers can include hardwood fibers, softwood fibers, and mixtures thereof.

The term “average fiber length” as used herein refers to an average length of fibers, fiber bundles and/or fiber-like materials determined by measurement utilizing microscopic techniques. A sample of at least 20 randomly selected fibers is separated from a liquid suspension of fibers. The fibers are set up on a microscope slide prepared to suspend the fibers in water. A tinting dye is added to the suspended fibers to color cellulose-containing fibers so they may be distinguished or separated from synthetic fibers. The slide is placed under a Fisher Stereomaster II Microscope—S19642/S19643 Series. Measurements of 20 fibers in the sample are made at 20× linear magnification utilizing a 0-20 mils scale and an average length, minimum and maximum length, and a deviation or coefficient of variation are calculated. In some cases, the average fiber length will be calculated as a weighted average length of fibers (e.g., fibers, fiber bundles, fiber-like materials) determined by equipment such as, for example, a Kajaani fiber analyzer Model No. FS-200, available from Kajaani Oy Electronics, Kajaani, Finland. According to a standard test procedure, a sample is treated with a macerating liquid to ensure that no fiber bundles or shives are present. Each sample is disintegrated into hot water and diluted to an approximately 0.001% suspension. Individual test samples are drawn in approximately 50 to 100 ml portions from the dilute suspension when tested using the standard Kajaani fiber analysis test procedure. The weighted average fiber length may be an arithmetic average, a length weighted average or a weight weighted average and may be expressed by the following equation:

$${\sum }_{{x}_{i} = 0}^{k} ( {x}_{i} * {n}_{i} ) / n$$

where
k=maximum fiber length
X_{f}=fiber length
nᵢ=number of fibers having length xi
n=total number of fibers measured.

One characteristic of the average fiber length data measured by the Kajaani fiber analyzer is that it does not discriminate between different types of fibers. Thus, the average length represents an average based on lengths of all different types, if any, of fibers in the sample.

As used herein the term “staple fibers” means discontinuous fibers made from synthetic polymers or regenerated cellulose, such as polypropylene, polyester, post consumer recycle (PCR) fibers, polyester, nylon, viscose, rayon, and the like, and those not hydrophilic may be treated to be hydrophilic. Staple fibers may be cut fibers or the like. Staple fibers can have cross-sections that are round, bicomponent, multicomponent, shaped, hollow, or the like.

The term “plied” or “bonded” or “coupled” refers herein to the joining, adhering, connecting, attaching, or the like, of two elements. Two elements will be considered plied, bonded or coupled together when they are joined, adhered, connected, attached, or the like, directly to one another or indirectly to one another, such as when each is directly bonded to intermediate elements. The plying, bonding or coupling of one element to another can occur via continuous or intermittent bonds.

As used herein, “binder fibers” are fibers that can bond to other fibers in a substrate using chemical, mechanical, or thermal means. The binder fibers may comprise thermally bondable fibers that, when heated, form thermal bonds with other fibers at their point of intersection. In one aspect, the binder fibers include a surface polymer having a lower melting temperature. For instance, the binder fibers can be made from a polymer, such as a polyolefin, having a melting temperature of less than 200 °C, such as less than 180 °C, such as less than 160 °C, such as less than 140 °C, such as less than 120 °C, such as less than 100 °C, and greater than 80 °C, such as greater than 90 °C. In one aspect, the binder fibers comprise conjugate fibers, such as bicomponent fibers. The conjugate fibers can have a core and sheath structure, including a core polymer surrounded by a sheath polymer. The core polymer can have a higher melting temperature than the sheath polymer. The core polymer can be selected for its strength and high melting point and the sheath polymer can be made from a polymer selected for its lower melting temperature. The core polymer, for instance, can have a melting temperature higher than the sheath polymer. In this manner, the sheath polymer, when subjected to heat, melts and bonds to other fibers within the web at intersecting points. The core polymer, however, allows the bicomponent binder fiber to retain its shape and provide strength.

As used herein, “synthetic polymer fibers” refers to fibers made from polymers that are not binder fibers. Synthetic polymer fibers can include polyester fibers, such as fibers made from a polyethylene terephthalate polymer. Other polymer synthetic fibers include polyolefin fibers, such as polyethylene fibers, polypropylene fibers, and fibers made from copolymers of the above.

As used herein, “saturation capacity” refers to the result of a saturation capacity test and is area normalized. The saturation capacity test is performed on an absorbent article using a table top saturation capacity tester as described herein. First, the dry sample mass is measured. Then, the samples are saturated for 20 minutes in a saline solution (0.9 wt % NaCl) and then allowed to drip dry for 1 minute. The samples are next placed body-facing side down on the mesh screen of the table top saturation capacity tester having 0.25 inch (6.4 mm) openings (commercially available from Taconic Plastics Inc. Petersburg, N.Y.) which, in turn, is placed on a vacuum box and covered with a flexible rubber dam material, such as a latex sheet. A vacuum of 3.5 kilopascals (0.5 pounds per square inch) is drawn in the vacuum box for a period of 5 minutes. The sample is then removed from the vacuum box and weighed to determine a saturated, or wet weight of the sample. If material, such as superabsorbent material or fiber, is drawn through the fiberglass screen while on the vacuum box, a screen having smaller openings should be used. Alternatively, a piece of a tea bag material (such as heat sealable tea bag material (grade 542, commercially available from the Kimberly-Clark Corporation)) can be placed between the material and the screen and the final value adjusted for the fluid retained by the tea bag material. The saturation capacity is the total weight of the wet sample minus the sample dry weight. The area-normalized saturation capacity is calculated by dividing the saturation capacity (in grams) by the area of the absorbent material (in square centimeters).

### DETAILED DESCRIPTION

It is to be understood by one of ordinary skill in the art that the present discussion is a description of exemplary embodiments only and is not intended as limiting the broader aspects of the present disclosure.

The present disclosure is directed to methods and systems that can produce nonwoven substrates. While the present disclosure provides examples of substrates manufactured through foam-forming, it is contemplated that the methods and apparatuses described herein may be utilized to benefit wet-laid and/or air-laid manufacturing processes.

Each example is provided by way of explanation and is not meant as a limitation. For example, features illustrated or described as part of one embodiment or figure can be used on another embodiment or figure to yield yet another embodiment. It is intended that the present disclosure include such modifications and variations.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. As used herein, the terminology of “first,” “second,” “third”, etc. does not designate a specified order, but is used as a means to differentiate between different occurrences when referring to various features in the present disclosure. Many modifications and variations of the present disclosure can be made without departing from the spirit and scope thereof. Therefore, the exemplary embodiments described herein should not be used to limit the scope of the invention.

In general, the present disclosure is directed to multi-layer substrates, particularly multi-layer substrates having excellent fluid handling properties including the ability to absorb large amounts of fluids. In one aspect, the multi-layer substrate can be made using a foam forming process which has been found to produce various advantages and benefits.

Multi-layer substrates made according to the present disclosure not only have good fluid absorbency characteristics, but also have excellent strength properties. In this manner, the substrates are easy to handle and manipulate. For instance, the multi-layer substrates made according to the present disclosure can be formed and then wound into a roll for later feeding to a process for producing absorbent articles. In accordance with the present disclosure, the multi-layer substrate of the present disclosure has at least two layers. One layer, an absorbent layer, contains significant amounts of superabsorbent materials for quickly absorbing and retaining fluids. The absorbent layer is combined with at least one outer layer. The at least one outer layer includes a binder, particularly binder fibers, that provide structure to the substrate without significantly impacting the thickness of the substrate. The at least one outer layer generally does not contain superabsorbent materials in any great amounts. In addition, the absorbent layer containing the superabsorbent materials is constructed so as to contain a minimal amount of binder fibers or no binder fibers. In this manner, the binder fibers do not interfere with the ability of the absorbent layer to absorb fluids and swell in an unrestricted manner. The multi-layer substrate of the present disclosure is particularly well suited for absorbent articles that are intended to absorb large amounts of fluids, such as diapers, other childcare products, and adult incontinence products.

By selecting the materials contained in each layer of the multi-layer substrate and by controlling the amount of the components contained in each layer, multi-layer substrates made according to the present disclosure can have an excellent balance of absorbency and strength. For example, regarding fluid handling properties, the multi-layer substrate of the present disclosure can display a 30 second AUL test result of greater than about 10 g/g, such as greater than about 11 g/g, such as greater than about 12 g/g, such as greater than about 13 g/g, such as greater than about 14 g/g, such as greater than about 15 g/g, such as greater than about 16 g/g, such as greater than about 17 g/g, such as greater than about 18 g/g, such as greater than about 19 g/g. The substrate can display a 30 second AUL test result of generally less than about 30 g/g. In addition, the substrate can display an area-normalized saturation capacity of greater than about 0.25 g/cm², such as greater than about 0.5 g/cm², such as greater than about 0.8 g/cm², such as greater than about 1.0 g, such as greater than about 1.2 g, such as greater than about 1.5 g/cm², and less than about 2.5 g/cm². The initial saturation capacity prior to being normalized can be greater than about 200 g, such as greater than about 225 g, such as greater than about 250 g, such as greater than about 275 g, such as greater than about 300 g, such as greater than about 325 g, such as greater than about 350 g, such as greater than about 375 g, such as greater than about 400 g, such as greater than about 425 g, such as greater than about 450 g, such as greater than about 475 g, such as greater than about 500 g. The absorbent capacity can be less than about 1,000 g.

In addition to the above fluid handling properties, the multi-layer substrate can also display a tensile strength in at least one direction of greater than about 2,000 gf/3 in. For example, the tensile strength of the substrate in one direction can be greater than about 2,500 gf/3 in, such as greater than about 3,000 gf/3 in, such as greater than about 3,500 gf/3 in, such as greater than about 4,000 gf/3 in, such as greater than about 4,500 gf/3 in, such as greater than about 5,000 gf/3 in, such as greater than about 5,500 gf/3 in, such as greater than about 6,000 gf/3 in, such as greater than about 6,500 gf/3 in, such as greater than about 7,000 gf/3 in, such as greater than about 7,500 gf/3 in, such as greater than about 8,000 gf/3 in, such as greater than about 8,500 gf/3 in, such as greater than about 9,000 gf/3 in, such as greater than about 9,500 gf/3 in, such as greater than about 10,000 gf/3 in, and less than about 20,000 gf/3 in. The normalized tensile strength can be greater than about 333 gf/in, such as greater than about 500 gf/in, such as greater than about 700 gf/in, such as greater than about 1,000 gf/in, such as greater than about 1,200 gf/in, such as greater than about 1,300 gf/in, such as greater than about 1,500 gf/in, such as greater than about 2,000 gf/in, such as greater than about 2,500 gf/in, such as greater than about 3,000 gf/in, and generally less than about 10,000 gf/in.

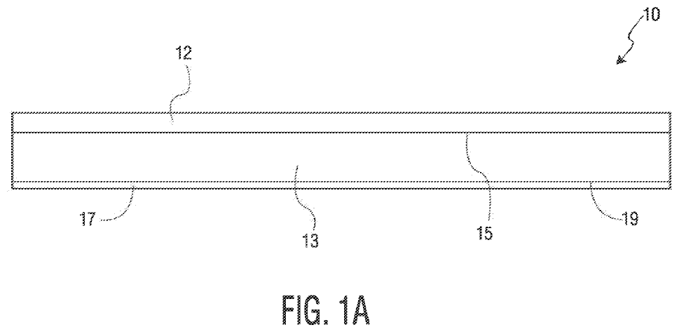

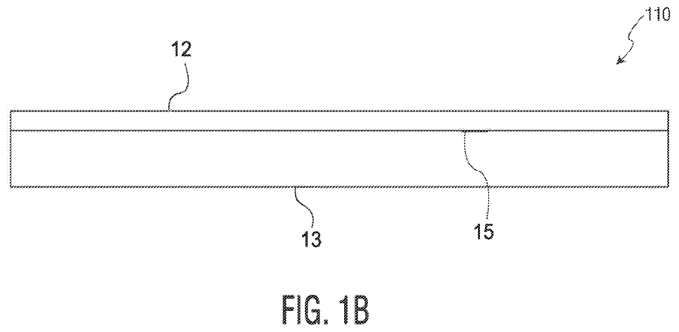

Referring to FIGS. 1A and 1B, for exemplary purposes only, examples of multi-layer absorbent substrates made in accordance with the present disclosure are shown. FIG. 1A illustrates a three-layer embodiment, while FIG. 1B illustrates a two-layer embodiment. In other embodiments, however, it should be understood that the absorbent substrate can contain more than three layers.

Referring to FIG. 1B, the two-layer substrate 110 includes a first layer 12 and a second layer 13. The second layer 13 is an absorbent layer that can contain significant amounts of superabsorbent materials. The first layer 12, on the other hand, can be constructed so as to provide strength while also being relatively thin and having high permeability for allowing fluids to quickly pass through the first layer 12 and be absorbed by the second layer 13.

As shown in FIG. 1B, the multi-layer substrate 110 can further include an interface 15 positioned between the first layer 12 and the second layer 13. In one aspect, at least some of the materials contained in the first layer 12 can be mixed with at least some of the materials in the second layer 13. For instance, fibers from the first layer 12 can be mixed with fibers and/or superabsorbent materials contained in the second layer 13. The interface 15 can provide the benefit of having some fiber distribution or other material distribution between each of the layers 12 and 13 that can provide intake benefits as well as some stabilization properties between the two layers.

In accordance with the present disclosure, the first layer 12 can be a top layer that is configured to face a wearer when the multi-layer substrate 110 is incorporated into an absorbent article. In one aspect, the first layer 12 can also be an intake layer designed to quickly allow fluids to be absorbed by the second layer 13. For example, in one embodiment, the first layer 12 can be a low-density layer with high permeability characteristics. In accordance with the present disclosure, the first layer 12 can also contain a binder, such as binder fibers, that provide strength and integrity to the overall substrate 110.

The binder contained within the first layer 12 can be any suitable binder material capable of binding adjacent fibers together. The binder, for instance, can be in powder form, such as a polyethylene powder. In one aspect, the binder can be water insoluble once dried on the multi-layer substrate. In other embodiments, the binder can be an adhesive material, such as a latex. The latex can be cationic or anionic to facilitate application to an adherence to the fibers contained within the substrate. Adhesives that may be used include anionic styrene-butadiene copolymers, polyvinyl acetate homopolymers, vinyl-acetate ethylene copolymers, vinyl-acetate acrylic copolymers, ethylene-vinyl chloride copolymers, ethylene-vinyl chloride-vinyl acetate terpolymers, acrylic polyvinyl chloride polymers, acrylic polymers, nitrile polymers, as well as other suitable anionic latex polymers known in the art. In one aspect, however, the multi-layer substrate of the present disclosure is formed without the use of adhesives in the first layer 12 and instead uses binder fibers.

Binder fibers that can be incorporated into the first layer 12 include mono-component fibers and multi-component fibers. The multi-component fibers, for instance, can include a core polymer surrounded by a sheath polymer. The sheath polymer can be comprised of a low melting thermoplastic polymer such as polyethylene. In one aspect, the binder fibers comprise bicomponent fibers containing a core polymer made from a polyester polymer or a polypropylene polymer. For example, the core polymer can be a polyethylene terephthalate polymer. The sheath polymer, on the other hand, can have a lower melting temperature than the core polymer and can comprise a polyolefin, such as polyethylene.

The binder fibers can have any suitable size and length. For instance, the binder fibers can have a length of between about 0.5 mm to about 50 mm, such as from about 0.75 mm to about 30 mm. In one aspect, the binder fibers have a length of from about 1 mm to about 25 mm. The binder fibers can have a size of from about 0.1 denier to about 10 denier. For instance, the size can be less than about 15 denier, such as less than about 10 denier, such as less than about 7 denier, such as ess than about 3 denier, such as less than about 2 denier, and greater than about 0.7 denier, such as greater than about 1 denier.

In one aspect, the first layer 12 can contain binder fibers in an amount from about 10% by weight to 100% by weight, including all increments of 1% therebetween. For example, the first layer 12 can contain binder fibers in an amount greater than about 20% by weight, such as in an amount greater than about 30% by weight, such as in an amount greater than about 40% by weight, such as in an amount greater than about 50% by weight, such as in an amount greater than about 60% by weight, such as in an amount greater than about 70% by weight, such as in an amount greater than about 80% by weight. The binder fibers can generally be present in an amount less than about 90% by weight, such as in an amount less than about 80% by weight, such as in an amount less than about 70% by weight.

In one aspect, the first layer 12 can contain more than one binder fiber. Each binder fiber, for instance, can vary in average fiber length and/or fiber size. In one aspect, for instance, the first layer 12 can include first binder fibers combined with second binder fibers. The first binder fibers, for instance, can have a fiber size larger than the second binder fibers and can be present in the top layer in an amount greater than the second binder fibers. The first binder fibers, for instance, can have a fiber size of greater than about 3 denier, such as greater than about 4 denier, such as greater than about 5 denier, and less than about 15 denier, such as less than about 10 denier, such as less than about 8 denier. The first binder fibers can be present in the top layer in an amount from about 10% by weight to about 55% by weight. For instance, the first binder fibers can be present in the top layer in an amount greater than about 20% by weight, such as in an amount greater than about 25% by weight, such as in an amount greater than about 30% by weight, such as in an amount greater than about 35% by weight, and in an amount less than about 65% by weight, such as in an amount less than about 60% by weight, such as in an amount less than about 55% by weight, such as in an amount less than about 50% by weight, such as in an amount less than about 45% by weight, such as in an amount less than about 40% by weight. The first binder fibers can provide for void volume and resiliency for fast fluid absorption.

The second binder fibers contained in the top layer can have a fiber size of less than about 3 denier, such as less than about 2.5 denier, and greater than about 0.3 denier, such as greater than about 0.8 denier, such as greater than about 1 denier. The second binder fibers can be present in the top layer in an amount greater than about 5% by weight, such as in an amount greater than about 8% by weight, and in an amount less than about 40% by weight, such as in an amount less than about 35% by weight, such as in an amount less than about 30% by weight, such as in an amount less than about 25% by weight, such as in an amount less than about 20% by weight. The second binder fibers can increase the strength of the substrate.

The first layer 12, although providing strength to the multi-layer substrate 110, can be relatively lightweight, thin and liquid permeable. In general, the basis weight of the first layer 12 can be from about 10 gsm to about 100 gsm, including all increments of 1 gsm therebetween. For instance, the basis weight can be less than about 90 gsm, such as less than about 80 gsm, such as less than about 70 gsm, such as less than about 60 gsm, such as less than about 50 gsm, such as less than about 40 gsm, such as less than about 30 gsm, such as less than about 20 gsm. The basis weight is generally greater than about 15 gsm, such as greater than about 20 gsm, such as greater than about 25 gsm, such as greater than about 30 gsm, such as greater than about 35 gsm, such as greater than about 40 gsm, such as greater than about 45 gsm, such as greater than about 50 gsm.

In addition to containing binder fibers, the first layer 12 can contain various other materials, including other fibers. In one aspect, for instance, the first layer 12 can contain polymer synthetic fibers. The polymer synthetic fibers, for instance, can be made from a polymer material and can be non-absorbent. As described above, in one aspect, the multi-layer substrate 110 can be produced using a foam forming process in which the fibers and other materials are suspended in a foam and then deposited onto a forming surface to form the multi-layer structure. Of advantage, the foam forming process can accommodate all different types of materials and fibers including polymer synthetic fibers. For example, the polymer synthetic fibers can have a bending stiffness that is substantially unimpacted by the presence of the forming fluid.

Examples of synthetic polymer fibers include polyolefin, polyester (PET), polyamide, polylactic acid, or other fiber forming polymers. Polyolefin fibers, such as polyethylene (PE) and polypropylene (PP), and polyethylene terephthalate fibers are particularly well suited for use in the present disclosure. In some embodiments, non-absorbent fibers can be recycled fibers, compostable fibers, and/or marine degradable fibers. In this regard, due to its very low levels of absorbency to water, water resistant fibers do not experience a significant change in bending stiffness upon contacting an aqueous fluid and therefore are capable of maintaining an open composite structure upon wetting. The fiber diameter of a fiber can contribute to enhanced bending stiffness. For example, a PET fiber has a higher bending stiffness than a polyolefin fiber whether in dry or wet states. The higher the fiber denier, the higher the bending stiffness a fiber exhibits. Water resistant fibers desirably have a water retention value (WRV) less than about 1 and still more desirably between about 0 and about 0.5. In certain aspects, it is desirable that the fibers, or at least a portion thereof, include non-absorbent fibers.

The synthetic and/or water resistant fibers can have fiber length greater than about 0.2 mm including, for example, having an average fiber length between about 0.5 mm and about 50 mm or between about 0.75 and about 30 mm or even between about 1 mm and about 25 mm. In one aspect, the synthetic polymer fibers can have an average fiber length of from about 4 mm to about 10 mm, such as from about 4 mm to about 8 mm.

The synthetic polymer fibers can have a fiber size of from about 0.3 denier to about 25 denier, including all increments of 0.1 denier therebetween. For instance, in one aspect, relatively fine fibers can be used having a denier of less than about 2, such as less than about 1.5, such as less than about 1, such as less than about 0.8, and greater than about 0.3, such as greater than about 0.5. Alternatively, the size of the polymer synthetic fibers can be from about 2 denier to about 20 denier. In one aspect, the size of the fibers can be from about 4 denier to about 8 denier. In another aspect, the size of the fibers can be from about 8 denier to about 15 denier, such as from about 9 denier to about 13 denier.

In some embodiments, the synthetic and/or water resistant fibers can have a crimped structure to enhance bulk generation capability of the foam formed fibrous substrate. For example, a PET crimped staple fiber may be able to generate a higher caliper (or result in a low sheet density) in comparison to a PET straight staple fiber with the same fiber diameter and fiber length.

For exemplary purposes, the first layer 12 can contain synthetic polymer fibers generally in an amount from about 1% by weight to about 80% by weight, including all increments of 1% by weight therebetween. For instance, the polymer synthetic fibers can be present in the first layer 12 in an amount greater than about 5% by weight, such as in an amount greater than about 10% by weight, such as in an amount greater than about 15% by weight, such as in an amount greater than about 20% by weight, such as in an amount greater than about 25% by weight, such as in an amount greater than about 30% by weight, such as in an amount greater than about 40% by weight, such as in an amount greater than about 50% by weight, such as in an amount greater than about 60% by weight. The polymer synthetic fibers can be present in the first layer 12 in an amount less than about 90% by weight, such as in an amount less than about 80% by weight, such as in an amount less than about 70% by weight, such as in an amount less than about 60% by weight, such as in an amount less than about 50% by weight, such as in an amount less than about 40% by weight, such as in an amount less than about 35% by weight, such as in an amount less than about 30% by weight, such as in an amount less than about 25% by weight, such as in an amount less than about 20% by weight, such as in an amount less than about 15% by weight.

In addition to or instead of polymer synthetic fibers, the first layer 12 can also contain cellulose fibers. Various different types of cellulose fibers can be incorporated into the first layer 12. In some embodiments, the fibers utilized can be conventional papermaking fibers such as wood pulp fibers formed by a variety of pulping processes, such as kraft pulp, sulfite pulp, bleached chemithermomechanical pulp (BCTMP), chemithermomechanical pulp (CTMP), pressure/pressure thermomechanical pulp (PTMP), thermomechanical pulp (TMP), thermomechanical chemical pulp (TMCP), and so forth. By way of example only, fibers and methods of making wood pulp fibers are disclosed in U.S. Pat. No. 4,793,898 to Laamanen et al.; U.S. Pat. No. 4,594,130 to Chang et al.; U.S. Pat. No. 3,585,104 to Kleinhart; U.S. Pat. No. 5,595,628 to Gordon et al.; U.S. Pat. No. 5,522,967 to Shet; and so forth. Further, the fibers may be any high-average fiber length wood pulp, low-average fiber length wood pulp, or mixtures of the same. Examples of suitable high-average length pulp fibers include softwood fibers, such as, but not limited to, northern softwood, southern softwood, redwood, red cedar, hemlock, pine (e.g., southern pines), spruce (e.g., black spruce), and the like. Examples of suitable low-average length pulp fibers include hardwood fibers, such as, but not limited to, eucalyptus, maple, birch, aspen, and the like.

Moreover, if desired, secondary fibers obtained from recycled materials may be used, such as fiber pulp from sources such as, for example, newsprint, reclaimed paperboard, and office waste. In some embodiments, refined fibers can be such that the total amount of virgin and/or high average fiber length wood fibers, such as softwood fibers, may be reduced.

Regardless of the origin of the wood pulp fiber, the wood pulp fibers preferably have an average fiber length greater than about 0.2 mm and less than about 3 mm, such as from about 0.35 mm and about 2.5 mm, or between about 0.5 mm to about 2.5 mm or even between about 0.7 mm and about 2.0 mm.

In addition, other cellulosic fibers that can be used in the present disclosure includes nonwoody fibers. As used herein, the term “non-wood fiber” generally refers to cellulosic fibers derived from non-woody monocotyledonous or dicotyledonous plant stems. Non-limiting examples of dicotyledonous plants that may be used to yield non-wood fiber include kenaf, jute, flax, ramie and hemp. Non-limiting examples of monocotyledonous plants that may be used to yield non-wood fiber include cereal straws (wheat, rye, barley, oat, etc.), stalks (corn, cotton, sorghum, Hesperaloe funifera, etc.), canes (bamboo, sisal, bagasse, etc.) and grasses (miscanthus. esparto, lemon, sabai, switchgrass, etc). In still other certain instances non-wood fiber may be derived from aquatic plants such as water hyacinth, microalgae such as Spirulina, and macroalgae seaweeds such as red or brown algae.

Still further, other cellulosic fibers for making substrates herein can include synthetic cellulose fiber types formed by spinning, including rayon in all its varieties, and other fibers derived from viscose or chemically-modified cellulose such as, for example, those available under the trade names LYOCELL and TENCEL.

Crosslinked cellulosic fibers, such as CMC 535, can also be used in forming materials 10, 110 described herein. Crosslinked cellulosic fibers can provide increased bulk and resiliency, as well as improved softness.

In some embodiments, the non-woody and synthetic cellulosic fibers can have fiber length greater than about 0.2 mm including, for example, having an average fiber size between about 0.5 mm and about 50 mm or between about 0.75 and about 30 mm or even between about 1 mm and about 25 mm. Generally speaking, when fibers of relatively larger average length are being used, it may often be advantageous to modify the amount and type of foaming surfactant. For example, in some embodiments, if fibers of relatively larger average length are being used, it may be beneficial to utilize relatively higher amounts of foaming surfactant in order to help achieve a foam with the required foam half life.

For exemplary purposes only, the first layer 12 can contain cellulose fibers generally in an amount from about 1% by weight to about 80% by weight, including all increments of 1% by weight therebetween. For instance, the cellulose fibers can be present in the first layer 12 in an amount greater than about 5% by weight, such as in an amount greater than about 10% by weight, such as in an amount greater than about 15% by weight, such as in an amount greater than about 20% by weight, such as in an amount greater than about 25% by weight, such as in an amount greater than about 30% by weight, such as in an amount greater than about 40% by weight, such as in an amount greater than about 50% by weight, such as in an amount greater than about 50% by weight. The cellulose fibers can be present in the first layer 12 in an amount less than about 90% by weight, such as in an amount less than about 80% by weight, such as in an amount less than about 70% by weight, such as in an amount less than about 60% by weight, such as in an amount less than about 50% by weight, such as in an amount less than about 40% by weight, such as in an amount less than about 35% by weight, such as in an amount less than about 30% by weight, such as in an amount less than about 25% by weight, such as in an amount less than about 20% by weight, such as in an amount less than about 15% by weight. The cellulose fibers can be a single type of fiber or can be a mixture of different cellulose fibers. For instance, the cellulose fibers incorporated into the first layer 12 can be regenerated cellulose fibers, pulp fibers including crosslinked pulp fibers, or mixtures thereof. In still another embodiment, the cellulose fibers can comprise cotton fibers alone or in combination with pulp fibers.

In one aspect, the first layer 12 can have a basis weight of greater than about 35 gsm, such as greater than about 40 gsm, such as greater than about 45 gsm, such as greater than about 50 gsm, and less than about 100 gsm, such as less than about 90 gsm, such as less than about 80 gsm. In this embodiment, the first layer 12 can contain from about 20% to about 80% binder fibers, such as from about 20% to about 60% binder fibers. The binder fibers can be combined with polymer synthetic fibers. The polymer synthetic fibers can be present generally in an amount greater than about 40% by weight, such as in an amount greater than about 50% by weight, such as in an amount greater than about 60% by weight, and in an amount less than about 90% by weight, such as in an amount less than about 80% by weight, such as in an amount less than about 70% by weight, such as in an amount less than about 60% by weight. In one aspect, the first layer 12 does not contain cellulose fibers.

In another aspect, the first layer 12 can have a basis weight of less than about 35 gsm, such as less than about 30 gsm, such as less than about 25 gsm, such as less than about 20 gsm, and greater than about 10 gsm, such as greater than about 15 gsm, such as greater than about 20 gsm. The first layer 12 can contain binder fibers in an amount from about 10% to about 80% by weight and can be combined with cellulose fibers, particularly pulp fibers and/or crosslinked cellulose fibers. The pulp fibers can be present in an amount greater than about 20% by weight, such as in an amount greater than about 30% by weight, such as in an amount greater than about 40% by weight, such as in an amount greater than about 50% by weight, and in an amount less than about 90% by weight, such as in an amount less than about 80% by weight, such as in an amount less than about 70% by weight, such as in an amount less than about 60% by weight. The first layer 12 can only contain binder fibers combined with cellulose fibers or can also contain polymer synthetic fibers. The polymer synthetic fibers can be present in the first layer 12 in this embodiment in an amount greater than about 5% by weight, such as in an amount greater than about 10% by weight, such as in an amount greater than about 15% by weight, such as in an amount greater than about 20% by weight, and generally in an amount less than about 60% by weight, such as in an amount less than about 40% by weight, such as in an amount less than about 20% by weight.

The absorbent layer 13 contained in the multi-layer substrate 110 is generally configured to absorb fluids, particularly liquids, and includes absorbent material. The absorbent material can include absorbent particles including fibers and/or other absorbent components. In one aspect, the second layer 13 has a size and mass capable of absorbing relatively large amounts of liquids so that the substrate 110 is well suited for being incorporated into absorbent articles such as diapers, child pants, adult incontinence products, and the like. For example, the second layer 13 or absorbent layer can have sufficient mass such that the multi-layer substrate 110 displays an area-normalized saturation capacity of greater than about 0.25 g/cm², such as greater than about 0.5 g/cm², such as greater than about 0.8 g/cm², such as greater than about 1.0 g/cm².

In one aspect, the second layer 13 is made primarily from superabsorbent materials (SAM). SAM is commonly provided in a particulate form and, in certain aspects, can comprise polymers of unsaturated carboxylic acids or derivatives thereof. In some forms, however, SAM can be configured in fiber form. These polymers are often rendered water insoluble, but water swellable, by crosslinking the polymer with a di- or polyfunctional internal crosslinking agent. These internally cross-linked polymers are at least partially neutralized and commonly contain pendant anionic carboxyl groups on the polymer backbone that enable the polymer to absorb aqueous fluids, such as body fluids. Typically, the SAM particles are subjected to a post-treatment to crosslink the pendant anionic carboxyl groups on the surface of the particle. SAMs are manufactured by known polymerization techniques, desirably by polymerization in aqueous solution by gel polymerization. The products of this polymerization process are aqueous polymer gels, i.e., SAM hydrogels that are reduced in size to small particles by mechanical forces, then dried using drying procedures and apparatus known in the art. The drying process is followed by pulverization of the resulting SAM particles to the desired particle size. Examples of superabsorbent materials include, but are not limited to, those described in U.S. Pat. No. 7,396,584 Azad et al., U.S. Pat. No. 7,935,860 Dodge et al., US2005/5245393 to Azad et al., US2014/09606 to Bergam et al., WO2008/027488 to Chang et al. and so forth.

In some embodiments involving SAM, the SAM may be treated by a water-soluble protective coating having a rate of dissolution selected such that the component is not substantially exposed to the aqueous liquid carrier until the highly-expanded foam has been formed and drying operations initiated that can remove the coating. Alternatively, in order to prevent or limit premature expansion during processing, the SAM may be introduced into the process at low temperatures.

Superabsorbent materials can be contained in the second layer 13 in an amount from about 20% to 100% by weight, including all increments of 1% by weight therebetween. The second layer 13, for example, can contain superabsorbent materials in an amount greater than about 30% by weight, such as in an amount greater than about 40% by weight, such as in an amount greater than about 50% by weight, such as in an amount greater than about 60% by weight, such as in an amount greater than about 70% by weight, such as in an amount greater than about 80% by weight, such as in an amount greater than about 90% by weight, such as in an amount greater than about 95% by weight. In various embodiments, the superabsorbent materials can be contained in the second layer 13 in an amount less than about 95% by weight, such as in an amount less than about 90% by weight, such as in an amount less than about 80% by weight, such as in an amount less than about 70% by weight, such as in an amount less than about 60% by weight. The second layer 13 can have a basis weight that is higher than the basis weight of the first layer 12. In one aspect, the basis weight of the second layer 13 is greater than about 200 gsm, such as greater than about 250 gsm, such as greater than about 300 gsm, such as greater than about 350 gsm, such as greater than about 400 gsm, such as greater than about 450 gsm, such as greater than about 500 gsm. The basis weight of the second layer 13 can be less than about 1,000 gsm, such as less than about 800 gsm, such as less than about 600 gsm.

In one aspect, the second layer 13 contains little to no binder fibers in order to allow the second layer 13 to absorb liquids and swell. For example, the second layer 13 can contain binder fibers in an amount less than about 20% by weight, such as in an amount less than about 5% by weight, such as in an amount less than about 4% by weight, such as in an amount less than about 3% by weight, such as in an amount less than about 2% by weight, such as in an amount less than about 1% by weight, such as in an amount less than about 0.5% by weight. In one embodiment, the second layer 13 is binder fiber-free or may only contain binder fibers that have migrated from the first layer 12 to the second layer 13 through the interface 15.

In addition to superabsorbent materials, the second layer 13 can also contain synthetic polymer fibers and/or cellulose fibers. When other fibers are present in the second layer 13, binder fibers can be present in the second layer in an amount less than about 5% by weight based upon the total weight of fibers contained in the layer, such as less than about 3% by weight based upon the total weight of fibers contained in the layer, such as less than about 1% by weight based upon the total weight of fibers contained in the layer.

In one aspect, the second layer 13 contains cellulose fibers. The cellulose fibers can be any of the cellulose fibers described above including regenerated cellulose fibers, cotton fibers, other natural cellulose fibers, pulp fibers, or mixtures thereof. The pulp fibers can be, for instance, softwood fibers, hardwood fibers, bast fibers, or mixtures thereof. The pulp fibers can comprise delignified cellulose fibers. In one aspect, pulp fibers are present in the second layer 13 that comprise crosslinked cellulose fibers or crosslinked pulp fibers. When present, cellulose fibers can be contained in the second layer 13 in an amount greater than about 5% by weight, such as in an amount greater than about 10% by weight, such as in an amount greater than about 15% by weight, such as in an amount greater than about 20% by weight, such as in an amount greater than about 25% by weight, such as in an amount greater than about 30% by weight, such as in an amount greater than about 35% by weight, such as in an amount greater than about 45% by weight, such as in an amount greater than about 50% by weight, such as in an amount greater than about 55% by weight, such as in an amount greater than about 60% by weight. One or more different types of cellulose fibers can be present in the second layer 13 generally in an amount up to 100% by weight and, in one aspect, in an amount less than about 80% by weight, such as in an amount less than about 70% by weight, such as in an amount less than about 60% by weight, such as in an amount less than about 50% by weight, such as in an amount less than about 40% by weight.

The second layer 13 can also contain polymer synthetic fibers. The polymer synthetic fibers can be used alone or in conjunction with cellulose fibers. The polymer synthetic fibers incorporated into the second layer 13 can be any of the polymer synthetic fibers described above including polyester fibers, polyolefin fibers, and the like. Although optional, when present in the second layer 13, the polymer synthetic fibers can be present generally in an amount greater than about 3% by weight of the second layer, such as greater than about 5% by weight, such as greater than about 10% by weight, such as greater than about 15% by weight, such as greater than about 20% by weight, such as greater than about 25% by weight, such as greater than about 30% by weight. The polymer synthetic fibers can be present in the second layer 13 generally in an amount less than about 60% by weight, such as in an amount less than about 50% by weight, such as in an amount less than about 40% by weight, such as in an amount less than about 30% by weight, such as in an amount less than about 20% by weight, such as in an amount less than about 15% by weight, such as in an amount less than about 10% by weight, such as in an amount less than about 5% by weight.

In one aspect, the second layer 13 can contain from about 25% to about 35% by weight PET fibers having a size of from about 5 denier to about 7 denier, from about 50% to about 70% by weight crosslinked cellulose pulp fibers and from about 5% to about 15% by weight binder fibers having a size of from about 0.8 denier to about 3 denier.

Referring now to FIG. 1A, another embodiment of a multi-layer substrate 10 made in accordance with the present disclosure is shown. Like reference numerals have been used to indicate similar elements. In particular, the multi-layer substrate 10 includes a first layer 12 adjacent to a second layer 13 and can have constructions similar to the corresponding layers described in FIG. 1B. An interface 15 is between the first layer 12 and the second layer 13. In this embodiment, the multi-layer substrate 10 further includes a third layer 17. As shown, the second layer 13 is positioned between the first layer 12 and the third layer 17. As shown in FIG. 1A, the substrate 10 further includes an interface 19 between the second layer 13 and the third layer 17. In one aspect, some of the materials contained in the second layer can mix with some of the materials contained in the third layer. The interface 19 can provide the benefit of having some fiber distribution between the second layer 13 and the third layer 17 that can provide enhanced stabilization properties between the two layers.

The third layer 17 and the first layer 12 can serve as containment layers for the second layer 13. In particular, the first layer 12 and the third layer 17 can be configured to be relatively thin and have a low basis weight while providing enough strength for handling and converting while adding minimal stiffness. In addition, the first layer 12 and the third layer 17 can contain the superabsorbent material included in the second layer 13 from migrating to a surface of the substrate. In this manner, the first layer 12 and the third layer 17 can improve the feel and comfort of the multi-layer substrate 10 by reducing any gritty feel that may occur when there is contact with superabsorbent materials. The first layer 12 and the third layer 17 can also prevent superabsorbent particles from escaping making the material easier to handle and process. In addition, the first layer 12 and the third layer 17 can be constructed to allow the second layer 13 to absorb fluids and swell without significant restrictions.

The third layer 17 as shown in FIG. 1A can generally have a relatively low basis weight. For instance, the third layer 17 can have a basis weight of less than about 40 gsm, such as less than about 30 gsm, such as less than about 25 gsm, such as less than about 20 gsm, such as less than about 15 gsm, such as even less than about 10 gsm. The basis weight of the third layer 17 can be greater than about 5 gsm, such as greater than about 10 gsm, such as greater than about 15 gsm.

The third layer 17 can contain binder fibers alone or in combination with cellulose fibers and/or polymer synthetic fibers. The binder fibers can be present in the third layer 17, for instance, in an amount of from about 20% to about 100% by weight, including all increments of 1% by weight therebetween. For instance, binder fibers, such as bicomponent fibers, can be present in the third layer 17 in an amount greater than about 20% by weight, such as in an amount greater than about 30% by weight, such as in an amount greater than about 40% by weight, and in an amount less than about 80% by weight, such as in an amount less than about 70% by weight, such as in an amount less than about 60% by weight, such as in an amount less than about 50% by weight, such as in an amount less than about 40% by weight. The binder fibers can have a size of less than about 6 denier, such as less than about 4 denier, such as less than about 3 denier, such as less than about 2 denier, such as less than about 1.5 denier, such as less than about 1 denier, and greater than about 0.3 denier.

In one aspect, the third layer 17 contains binder fibers in combination with cellulose fibers, particularly pulp fibers. The pulp fibers can be softwood fibers, hardwood fibers, or combinations thereof. In one aspect, the pulp fibers are crosslinked pulp fibers. The cellulose fibers can be present in the third layer 17 in an amount of greater than about 10% by weight, such as in an amount greater than about 20% by weight, such as in an amount greater than about 30% by weight, such as in an amount greater than about 40% by weight, such as in an amount greater than about 50% by weight, such as in an amount greater than about 60% by weight, such as in an amount greater than about 70% by weight, such as in an amount greater than about 80% by weight. The cellulose fibers can be present in the third layer 17 in an amount less than about 90% by weight, such as in an amount less than about 80% by weight, such as in an amount less than about 70% by weight, such as in an amount less than about 60% by weight, such as in an amount less than about 50% by weight. Cellulose fibers can improve the wicking properties of the layer and can produce layers at very low basis weights while being able to contain the superabsorbent materials within the second layer 13.

Instead of or in addition to cellulose fibers, the third layer 17 can also contain polymer synthetic fibers. The polymer synthetic fibers can be any of the fibers described above, such as polyester fibers or polyolefin fibers. When included within the third layer 17, the synthetic polymer fibers can be present in an amount greater than about 3% by weight, such as in an amount greater than about 5% by weight, such as in an amount greater than about 10% by weight, such as in an amount greater than about 20% by weight, such as in an amount greater than about 30% by weight, such as in an amount greater than about 40% by weight, such as in an amount greater than about 50% by weight, such as in an amount greater than about 60% by weight, and generally in an amount less than about 80% by weight, such as in an amount less than about 70% by weight, such as in an amount less than about 60% by weight, such as in an amount less than about 50% by weight, such as in an amount less than about 40% by weight, such as in an amount less than about 30% by weight, such as in an amount less than about 25% by weight.

In one aspect, the third layer 17 can be constructed to be relatively liquid impermeable, which can be contrary to the construction of the first layer 12. The third layer 17, for instance, can be configured to protect the absorbent substrate 10 from not only losing superabsorbent materials but also can be configured to prevent the multi-layer substrate 10 from allowing liquid migration through the third layer 17 once the second layer 13 has absorbed liquids.

In other embodiments, the third layer 17 can be constructed to be liquid permeable. For instance, the third layer 17 can include three-dimensional synthetic fibers, such as crimped synthetic fibers, that can provide larger pore sizes for increased bulk and improved intake.

In addition to the materials described above to form the different layers in the multi-layer substrate, the multi-layer substrate can also contain various other additives and components. For example, wet strength additives can be added during formation of the substrate in order to help improve the relative strength of the multi-layer substrate.

Such strength additives suitable for use with paper making fibers and the manufacture of paper tissue are known in the art. Temporary wet strength additives may be cationic, nonionic or anionic. Examples of such temporary wet strength additives include PAREZ™ 631 NC and PAREZ (R) 725 temporary wet strength resins that are cationic glyoxylated polyacrylamides available from Cytec Industries, located at West Paterson, N.J. These and similar resins are described in U.S. Pat. No. 3,556,932 to Coscia et al. and U.S. Pat. No. 3,556,933 to Williams et al. Additional examples of temporary wet strength additives include dialdehyde starches and other aldehyde containing polymers such as those described in U.S. Pat. No. 6,224,714 to Schroeder et al.; U.S. Pat. No. 6,274,667 to Shannon et al.; U.S. Pat. No. 6,287,418 to Schroeder et al.; and U.S. Pat. No. 6,365,667 to Shannon et al., and so forth.

Permanent wet strength agents comprising cationic oligomeric or polymeric resins may also be used in the present disclosure. Polyamide-polyamine-epichlorohydrin type resins such as KYMENE 557H sold by Solenis are the most widely used permanent wet-strength agents and are suitable for use in the present disclosure. Such materials have been described in the following U.S. Pat. No. 3,700,623 to Keim; U.S. Pat. No. 3,772,076 to Keim; U.S. Pat. No. 3,855,158 to Petrovich et al.; U.S. Pat. No. 3,899,388 to Petrovich et al.; U.S. Pat. No. 4,129,528 to Petrovich et al.; U.S. Pat. No. 4,147,586 to Petrovich et al.; U.S. Pat. No. 4,222,921 to van Eenam and so forth. Other cationic resins include polyethylenimine resins and aminoplast resins obtained by reaction of formaldehyde with melamine or urea. Permanent and temporary wet strength resins may be used together in the manufacture of composite cellulosic products of the present disclosure. Further, dry strength resins may also optionally be applied to the composite cellulosic webs of the present disclosure. Such materials may include, but are not limited to, modified starches and other polysaccharides such as cationic, amphoteric, and anionic starches and guar and locust bean gums, modified polyacrylamides, carboxymethylcellulose, sugars, polyvinyl alcohol, chitosan, and the like.

When a wet or dry strength additive is used, it is preferable to select such an additive to be compatible with the foam agent used for the foam process. For example, when a strength additive is a cationic resin, due to incompatibility between a cationic and an anionic substance, a cationic surfactant is preferably used as a foam agent, or vice versa. A non-ionic surfactant is usually compatible with any cationic and anionic strength additives.

If used, such wet and dry strength additives can comprise between about 0.01 and about 5% of the dry weight of cellulose fibers contained in the multi-layer substrate. In certain embodiments, the strength additives can comprise between about 0.05% and about 2% of the dry weight of cellulose fibers or even between about 0.1% and about 1% of the dry weight of cellulose fibers.

Still other additional components may be added to multi-layer substrate materials. For materials that are formed utilizing foam forming processes, other additional components should be reviewed as to ensure they do not significantly interfere with the formation of the foam, the hydrogen bonding as between the cellulosic fibers or other desired properties of the material. As examples, additional additives may include one or more pigments, opacifying agents, anti-microbial agents, pH modifiers, skin benefit agents, odor absorbing agents, fragrances, thermally expandable microspheres, foam particles (such as, pulverized foam particles), and so forth as desired to impart or improve one or more physical or aesthetic attributes. In certain embodiments, the multi-layer substrate may include skin benefit agents such as, for example, antioxidants, astringents, conditioners, emollients, deodorants, external analgesics, film formers, humectants, hydrotropes, pH modifiers, surface modifiers, skin protectants, and so forth.

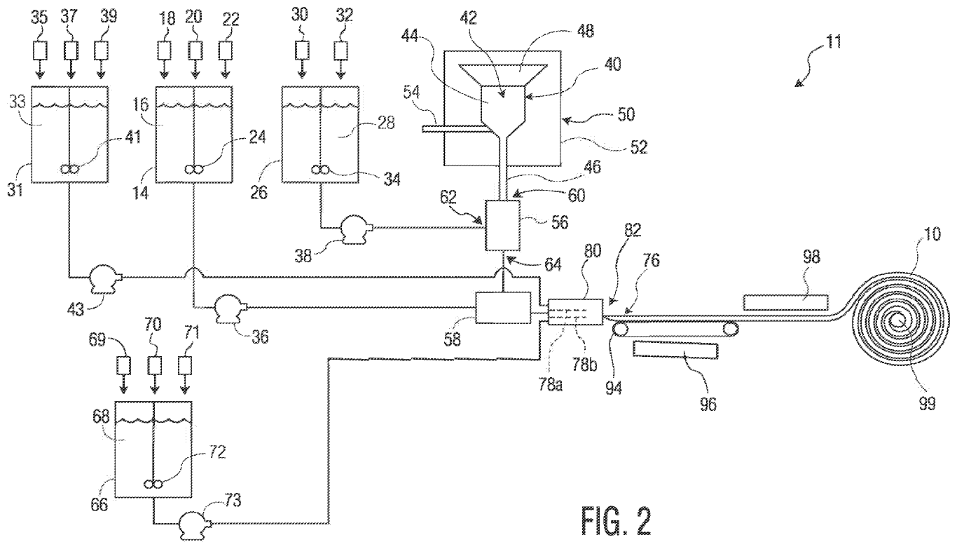

Multi-layer substrates, as described herein can be preferably formed through a foam forming process. FIG. 2 provides a schematic of an exemplary apparatus 11 that can be used as part of a foam forming process to manufacture a multi-layer substrate 10 that is a foam formed product. The apparatus 11 of FIG. 2 can include a first tank 14 configured for holding a first fluid supply 16. In some embodiments, the first fluid supply 16 can be a foam. The first fluid supply 16 can include a fluid provided by a supply of fluid 18. In some embodiments, the first fluid supply 16 can include a plurality fibers provided by a supply of fibers 20, and preferably includes at least some absorbent fibers. However, in other embodiments, the first fluid supply 16 can be free from a plurality of fibers altogether. The first fluid supply 16 can also include a surfactant provided by a supply of surfactant 22.

In some embodiments, the first tank 14 can include a mixer 24, as will be discussed in more detail below. The mixer 24 can mix (e.g., agitate) the first fluid supply 16 to mix the fluid, fibers (if present), and surfactant with air, or some other gas, to create a foam. The mixer 24 can also mix the foam with fibers (if present) to create a foam suspension of fibers in which the foam holds and separates the fibers to facilitate a distribution of the fibers within the foam (e.g., as an artifact of the mixing process in the first tank 14). Uniform fiber distribution can promote desirable absorbent material 10 including, for example, strength and the visual appearance of quality.

The apparatus 11 can also include a second tank 26 configured for holding a second fluid supply 28. In some embodiments, the second fluid supply 28 can be a foam. The second fluid supply 28 can include a fluid provided by a supply of fluid 30 and a surfactant provided by a supply of surfactant 32. In some preferred embodiments, such as depicted in FIG. 2, the second fluid supply 28 is free from fibers. In other embodiments, the second fluid supply 28 can include a plurality of fibers in addition to or as an alternative to the fibers being present in the first fluid supply 16. In some embodiments, the second tank 26 can include a mixer 34. The mixer 34 can mix the second fluid supply 28 to mix the fluid and surfactant with air, or some other gas, to create a foam.

In some embodiments, the apparatus 11 can also include a third tank 31 configured for holding a third fluid supply 33. In some embodiments, the third fluid supply 33 can be a foam. The third fluid supply 33 can include a fluid provided by a supply of fluid 35 and a plurality of fibers provided by a supply of fibers 37, and preferably includes at least some synthetic fibers. The third fluid supply 33 can also include a surfactant provided by a supply of surfactant 39. In some embodiments, the third tank 31 can include a mixer 41. The mixer 41 can mix the third fluid supply 33 to mix the fluid and surfactant with air, or some other gas, to create a foam.

In some embodiments, the apparatus 11 can also include a fourth tank 66 configured for holding a fourth fluid supply 68. In some embodiments, the fourth fluid supply 68 can be a foam. The fourth fluid supply 68 can include a fluid provided by a supply of fluid 69 and a plurality of fibers provided by a supply of fibers 70. The fourth fluid supply 68 can also include a surfactant provided by a supply of surfactant 71. In some embodiments, the fourth tank 66 can include a mixer 72. The mixer 72 can mix the fourth fluid supply 68 to mix the fluid and surfactant with air, or some other gas, to create a foam.

In tanks 14, 26, 31, 66 the first fluid supply 16, the second fluid supply 28, the third fluid supply 33, and the fourth fluid supply 68, respectively, can be acted upon to form a foam. In some embodiments, the foaming fluid and other components are acted upon so as to form a porous foam having an air content greater than about 50% by volume and desirably an air content greater than about 60% by volume. In certain aspects, the highly-expanded foam is formed having an air content of between about 60% and about 95% and in further aspects between about 65% and about 85%. In certain embodiments, the foam may be acted upon to introduce air bubbles such that the ratio of expansion (volume of air to other components in the expanded stable foam) is greater than 1:1 and in certain embodiments the ratio of air:other components can be between about 1.1:1 and about 20:1 or between about 1.2:1 and about 15:1 or between about 1.5:1 and about 10:1 or even between about 2:1 and about 5:1.

The foam can be generated by one or more means known in the art. Examples of suitable methods include, without limitation, aggressive mechanical agitation such as by mixers 24, 34, 41, 72 injection of compressed air, and so forth. Mixing the components through the use of a high-shear, high-speed mixer is particularly well suited for use in the formation of the desired highly-porous foams. Various high-shear mixers are known in the art and believed suitable for use with the present disclosure. High-shear mixers typically employ a tank holding the foam precursor and/or one or more pipes through which the foam precursor is directed. The high-shear mixers may use a series of screens and/or rotors to work the precursor and cause aggressive mixing of the components and air.

In a particular embodiment, the first tank 14, the second tank 26, the third tank 31, and/or the fourth tank 66 is provided having therein one or more rotors or impellors and associated stators. The rotors or impellors are rotated at high speeds in order to cause flow and shear. Air may, for example, be introduced into the tank at various positions or simply drawn in by the action of the mixers 24, 34, 41, 72. While the specific mixer design may influence the speeds necessary to achieve the desired mixing and shear, in certain embodiments suitable rotor speeds may be greater than about 500 rpm and, for example, be between about 1000 rpm and about 6000 rpm or between about 2000 rpm and about 4000 rpm. In other embodiments, suitable rotor speeds may be less than 500 rpm.

In addition, it is noted the foaming process can be accomplished in a single foam generation step or in sequential foam generation steps for the first tank 14, the second tank 26, the third tank 31, and/or the fourth tank 66. For example, in one embodiment, all of the components of the first fluid supply 16 in the first tank 14 (e.g., the supply of the fluid 18, fibers 20, and surfactant 22) may be mixed together to form a slurry from which a foam is formed. Alternatively, one or more of the individual components may be added to the foaming fluid, an initial mixture formed (e.g. a dispersion or foam), after which the remaining components may be added to the initially foamed slurry and then all of the components acted upon to form the final foam. In this regard, the fluid 18 and surfactant 22 may be initially mixed and acted upon to form an initial foam prior to the addition of any solids. Fibers, if desired, may then be added to the water/surfactant foam and then further acted upon to form the final foam. As a further alternative, the fluid 18 and fibers 20, such as a high density cellulose pulp sheet, may be aggressively mixed at a higher consistency to form an initial dispersion after which the foaming surfactant 22, additional water and other components, such as synthetic fibers, are added to form a second mixture which is then mixed and acted upon to form the foam.

The foam density of the foam forming the first fluid supply 16 in the first tank 14, the foam forming the second fluid supply 28 in the second tank 26, the third fluid supply 33 in the third tank 31, and/or the fourth fluid supply 68 in the fourth tank 66 can vary depending upon the particular application and various factors, such as the fiber stock used. In some implementations, for example, the foam density of the foam can be greater than about 100 g/L, such as greater than about 250 g/L, such as greater than about 300 g/L. The foam density is generally less than about 800 g/L, such as less than about 500 g/L, such as less than about 400 g/L, such as less than about 350 g/L. In some implementations, for example, a lower density foam is used having a foam density of generally less than about 350 g/L, such as less than about 340 g/L, such as less than about 330 g/L.

The apparatus 11 can also include a first pump 36, a second pump 38, third pump 43, and fourth pump 73. The first pump 36 can be in fluid communication with the first fluid supply 16 and can be configured for pumping the first fluid supply 16 to transfer the first fluid supply 16. The second pump 38 can be in fluid communication with the second fluid supply 28 and can be configured for pumping the second fluid supply 28 to transfer the second fluid supply 28. The third pump 43 can be in fluid communication with the third fluid supply 33 and can be configured for pumping the third fluid supply 33 to transfer the third fluid supply 33. The fourth pump 73 can be in fluid communication with the fourth fluid supply 68 and can be configured for pumping the fourth fluid supply 68 to transfer the fourth fluid supply 68. In some embodiments, the first pump 36, the second pump 38, the third pump 43, and/or the fourth pump 73 can be a progressive cavity pump or a centrifugal pump, however, it is contemplated that other suitable types of pumps can be used.

As depicted in FIG. 2, the apparatus 11 can also include a component feed system 40. The component feed system 40 can be used to deliver a supply of component 44, if one is desired for the multi-layer substrate 10, by delivering the component 44 to one or more fluid supply 16, 28, 33, 68 or directly to the headbox 80. One exemplary component feed system 40 that can be used can include a component supply area 42 for receiving a supply of a component. The component feed system 40 can also include an outlet conduit 46. The component feed system 40 can also include a hopper 48. The hopper 48 can be coupled to the component supply area 42 and can be utilized for refiling the supply of the component 44 to the component supply area 42.

In some embodiments, the component feed system 40 can include a bulk solids pump. Some examples of bulk solids pumps that may be used herein can include systems that utilize screws/augers, belts, vibratory trays, rotating discs, or other known systems for handling and discharging the supply of the component 44. Other types of feeders can be used for the component feed system 40, such as, for example, an ingredient feeder, such as those manufactured by Christy Machine & Conveyor, Fremont, Ohio. The component feed system 40 can also be configured as a conveyor system in some embodiments.

In some embodiments, the component feed system 40 can also include a pressure control system 50. In some embodiments, the pressure control system 50 can include a housing 52. The housing 52 can form a pressurized seal volume around the component feed system 40. In other embodiments, the pressure control system 50 can be formed as an integral part to the structure component feed system 40 itself, such that a separate housing 52 surrounding the component feed system 40 may not be required. The pressure control system 50 can also include a bleed orifice 54 in some embodiments.

The supply of the component 44 can be in the form of a particulate and/or a fiber and/or a powder. In one embodiment as described herein, the supply of the component 44 can be superabsorbent material (SAM) in particulate form. In some embodiments, SAM can be in the form of a fiber. Of course, other types of components, as previously discussed, are also contemplated as being utilized in the apparatus 11 and methods for forming an absorbent material 10 as described herein. The component feed system 40 as described herein can be particularly beneficial for a supply of component 44 that is most suitably maintained in a dry environment with minimal of exposure to fluid or foam utilized in the apparatus 11 and methods described herein.

The apparatus 11 can also include a first mixing junction 56 and a second mixing junction 58. In preferred embodiments, the first mixing junction 56 can be an eductor (also commonly referred to as a jet pump). The first mixing junction 56 can be in fluid communication with the outlet conduit 46 of the component feed system 40 and in fluid communication with the second fluid supply 28. The first mixing junction 56 can include a first inlet 60 and a second inlet 62. The first inlet 60 can be in fluid communication with the supply of the component 44 via the outlet conduit 46. The second inlet 62 can be in fluid communication with the second fluid supply 28. The first mixing junction 56 can also include a discharge 64. In preferred embodiments, the first mixing junction 56 can be configured as a co-axial eductor with the axis of the first inlet 60 being co-axial with the axis of the outlet conduit 46 that provides the supply of the component 44. The first mixing junction 56 can also be configured such that the discharge axis of the discharge 64 is co-axial with the outlet axis of the outlet conduit 46. As such, the first mixing junction 56 can be configured such that the axis of the first inlet 60 can be co-axial with the axis of the discharge 64 of the first mixing junction 56. The second inlet 62 providing the second fluid supply 28 to the first mixing junction 56 can be set up to enter the first mixing junction 56 on a side of the first mixing junction 56.

When configured as an eductor, the first mixing junction 56 can mix the supply of the component 44 from the component feed system 40 with the second fluid supply 28. By transferring the second fluid supply 28 into the first mixing junction 56 at the second inlet 62 and through the first mixing junction 56, the second fluid supply 28 provides a motive pressure to the supply of the component 44. The motive pressure can create a vacuum on the supply of the component 44 and the component feed system 40 to help draw the supply of the component 44 to mix and be entrained in the second fluid supply 28. In some embodiments, the motive pressure can create a vacuum on the supply of the component 44 of less than 1.5 in Hg, however, in other embodiments, the motive pressure could create a vacuum on the supply of the component 44 of 5 in. Hg or more, or 10 in Hg or more.

The pressure control system 50 can help manage proper distribution and entrainment of the supply of the component 44 to the second fluid supply 28. For example, when the second fluid supply 28 creates a motive pressure on the component feed system 40, the vacuum pulling on the supply of the component 44 may cause additional air to be entrained in the second fluid supply 28. In some circumstances, entraining additional air in the second fluid supply 28 may be desired, however, in other circumstances, it may be desirable to control the gas content of the second fluid supply 28 while inputting the supply of the component 44 to the second fluid supply 28 at the first mixing junction 56. For example, in some circumstances where the second fluid supply 28 is a foam, the amount of gas content in the foam may be desired to be kept relatively fixed as the foam passes through the first mixing junction 56. Thus, the pressure control system 50 can control the pressure on the component feed system 40 to help counteract the motive pressure on the supply of the component 44 and the component feed system 40 created by the second fluid supply 28.

In some embodiments, the pressure control system 50 can include sealing off the component feed system 40. For example, as discussed above, the pressure control system 50 can include a housing 52 to provide a seal on the component feed system 40. Sealing the component feed system 40 can help to prevent additional air entrainment in the second fluid supply 28 when the supply of the component 44 is introduced into the second fluid supply 28 in the first mixing junction 56.

However, in some embodiments, it may be beneficial to also include additional capability to the pressure control system 50. For example, in some embodiments, the pressure control system 50 can include a bleed orifice 54. The bleed orifice 54 can be configured to bleed-in pressure, such as atmospheric air pressure, to provide additional pressure control of the component feed system 40. It has been discovered that by providing a bleed-in orifice 54 to provide some bleed-in of atmospheric air pressure to the component feed system 40, back-splashing of the second fluid supply 28 in the first mixing junction 56 can be reduced or eliminated. Reducing back-splashing of the second fluid supply 28 in the first mixing junction 56 can help prevent the component feed system 40 from becoming clogged or needing to be cleaned, especially where the component feed system 40 may be delivering a dry component, such as particulate SAM.

Additionally or alternatively, the pressure control system 50 can be configured to provide additional positive pressure to prevent back-filling of the component feed system 40 in some circumstances, such as if a downstream obstruction occurs in the apparatus 11 beyond the first mixing junction 56. In such a case of an obstruction creating an increased pressure, the second fluid supply 28 may have a desire to back-fill the component feed system 40. Back-filling of fluid into the component feed system 40 can be detrimental to processing, especially where the supply of the component 44 is a component best kept in dry conditions, such as SAM. A pressure control system 50 configured to be able to provide positive pressure to the component feed system 40 can help prevent such back-filling of the component feed system 40.

It is also contemplated that other additional aspects of a pressure control system 50 could be utilized to maintain the pressure to a suitable level for the component feed system 40, including, but not limited to, supplying vacuum to the component feed system 40 in addition to or alternative to the air bleed-in at the bleed orifice 54 and/or the positive pressure described above.

The first mixing junction 56 can also provide pressure control on the transfer of the second fluid supply 28 including the component 44 as it exits the discharge 64 of the first mixing junction 56 as compared to when the second fluid supply 28 enters the first mixing junction 56. The second fluid supply 28 can be transferred at a second fluid pressure prior to the first mixing junction 56. The second fluid supply 28 including the component from the supply of the component 44 can exit the discharge 64 of the first mixing junction 56 at a discharge pressure. The pressure difference between the second fluid pressure prior to the first mixing junction 56 and the discharge pressure can be controlled. In some embodiments, this pressure difference can be controlled by varying the flow rate of the second fluid supply 28 or through the positioning of the outlet conduit 46 in the first mixing junction 56. In some embodiments, it is preferable to control the pressure difference between the second fluid pressure prior to the first mixing junction 56 and the discharge pressure to be less than or equal to 5 pounds per square inch.

It is to be noted that while a single outlet conduit 46 of the component feed system 40 and a single first mixing junction 56 is illustrated in FIG. 2, it is contemplated that the outlet conduit 46 can be split into two or more conduits to feed two or more first mixing junctions 56 for mixing the supply of the component 44 with the second fluid supply 28. In such a configuration, the second fluid supply 28 can include as many conduits as there are first mixing junctions 56. By having more than one outlet conduit 46 and more than one first mixing junction 56 to mix the supply of the component 44 with the second fluid supply 28, a greater flow rate of the second fluid supply 28 including the component from the supply of the component 44 can be achieved.

Referring to FIG. 2, the apparatus 11 can include a second mixing junction 58 in some embodiments. The second mixing junction 58 can provide the functionality of mixing the second fluid supply 28 including the component from the supply of the component 44 with the first fluid supply 16. As the second fluid supply 28 including the component from the supply of the component 44 exits the discharge 64 of the first mixing junction 56 it can be transferred to the second mixing junction 58. The first fluid supply 16 can be delivered to the second mixing junction 58 by the first pump 36. The second mixing junction 58 can mix the first fluid supply 16 and any of its components (e.g., fluid 18, fibers 20, surfactant 22) with the second fluid supply 28 and any of its components (e.g., fluid 30, surfactant 32) and the component from the supply of the component 44 to deliver the mixture of the first fluid supply 16, the second fluid supply 28, and the component 44 to a headbox 80.

Alternatively, in some embodiments, a second mixing junction 58 can be omitted from the apparatus 11 and the second fluid supply 28 including the component from the supply of the component 44 can be delivered to headbox 80.

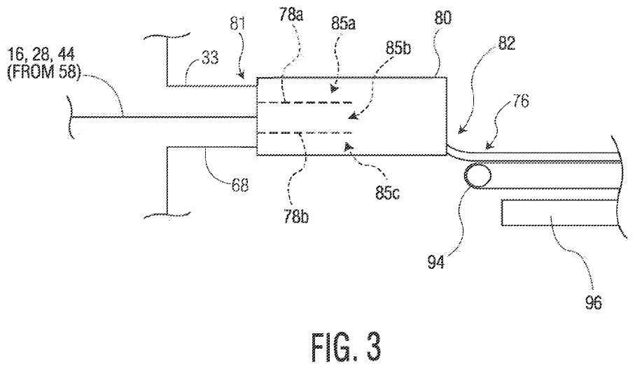

As illustrated in FIGS. 2 and 3, the headbox 80 can include one or more z-directional dividers 78a, 78b for separating different inputs to the headbox 80 in forming different layers of the absorbent material 10. The third fluid supply 33 and any of its components (e.g., fluid 35, fibers 37, surfactant 39) can be delivered to the inlet 81 of the headbox 80 via the third pump 43 and can be delivered above the first z-directional divider 78a in a first z-directional layer 85a of the headbox 80. The output of the second mixing junction 58 including the mixture of the first fluid supply 16 and any of its components (e.g., fluid 18, fibers 20, surfactant 22), the second fluid supply 28 and any of its components (e.g., fluid 30, surfactant 32), and the component 44 can be delivered to the inlet 81 of the headbox 80 below the first z-directional divider 78a and above the second z-directional divider 78b in a second z-directional layer 85b of the headbox 80. The fourth fluid supply 68 and any of its components (e.g., fluid 69, fibers 70, surfactant 71) can be delivered to the inlet 81 of the headbox 80 via the fourth pump 73 and can be delivered below the second z-directional divider 78b in a third z-directional layer 85c of the headbox 80. Such a configuration of two z-directional dividers 78a, 78b can be beneficial for forming a three-layered substrate 10, such as described above and illustrated in FIG. 1A. Of course, the two layered substrate 110, as described herein could be formed through a headbox 80 including a single z-directional divider 78a that provides a first z-directional layer 85a and a second z-directional layer 85b of the headbox 80. Further, in some embodiments, the headbox 80 need not include any z-directional dividers 78a, 78b, which may be particularly beneficial if further mixing of fibers and/or components within the headbox 80 is desired.

The headbox 80 can provide a resultant slurry 76 to a forming surface 94. The forming surface 94 can be a foraminous sheet, such as a woven belt or screen, or any other suitable surface for accepting the resultant slurry 76.

The apparatus 11 can also include a dewatering system 96 that can be configured to remove liquid from the resultant slurry 76 (e.g., forming fluid) on the forming surface 94. In some embodiments, the dewatering system 96 can be configured to provide a vacuum to the resultant slurry 76 to pull liquid from the resultant slurry 76, and in doing so, can turn the resultant slurry 76 including the plurality of fibers 20 and the component 44, if present, into an multi-layer substrate 10. In some embodiments, the dewatering system 96 can begin dewatering on fibers and/or components as they are still within the headbox 80.

Dewatering systems 96 drawing liquid from the resultant slurry 76 can also unintentionally draw components 44 (such as particulate SAM) through the forming surface 94, and/or cause components 44 to become lodged in the forming surface 94. Not only can this cause substrates 10 to be formed that do not include intended amounts of the component 44, but components 44 becoming lodged in the forming surface 94 and/or being drawn through the forming surface 94 can cause processing issues, including, but not limited to, reduced dewatering and/or increased demands for drying of the resultant slurry 76, machine down-time for cleaning, and increased complexity for dewatered liquid by including such components 44. Forming a multi-layer substrate 10 including components 44 in a fluid, such as foam forming, can exacerbate the problem of component 44 movement in the resultant slurry 76 in comparison to dry forming techniques, such as air-laid formation techniques or adhesive-based techniques.

Forming a third layer 17 as part of the substrate 10 that is directly against the forming surface 94 can help protect the components 44 of the substrate 12 (such as SAM in the absorbent layer 13). The third layer 17 can protect the components 44 of the substrate 10 from the forming surface to help ensure the components 44 remain in the substrate 10, or at least reduce the possibility for the components 44 to become lodged in the forming surface 94 or be drawn through the forming surface 94. Additionally, the third layer 17 can help retain components 44 within the absorbent material 10 as it is potentially transported for further processing and/or use in other products in which the multi-layer substrate 10 may be incorporated within, such as personal care absorbent articles. Forming the third layer 17 inline as a layered composite with the absorbent layer 13 where at least some fibers of the third layer 17 are mixed with at least some of the fibers of the absorbent layer 13 at the interface 19, eliminates the need for additional processing to form a composite absorbent substrate 10, such as the use of adhesive to couple a separate third layer 17 to an absorbent layer 13. Eliminating adhesive can result in reduced processing equipment and raw material cost and can also lead to improved fluid handling properties of the absorbent substrate 10. Additionally, forming a third layer 17 as part of the substrate 10 can also provide improved integrity and tensile strength for the absorbent material 10 providing enhanced processing capability of the substrate 10.

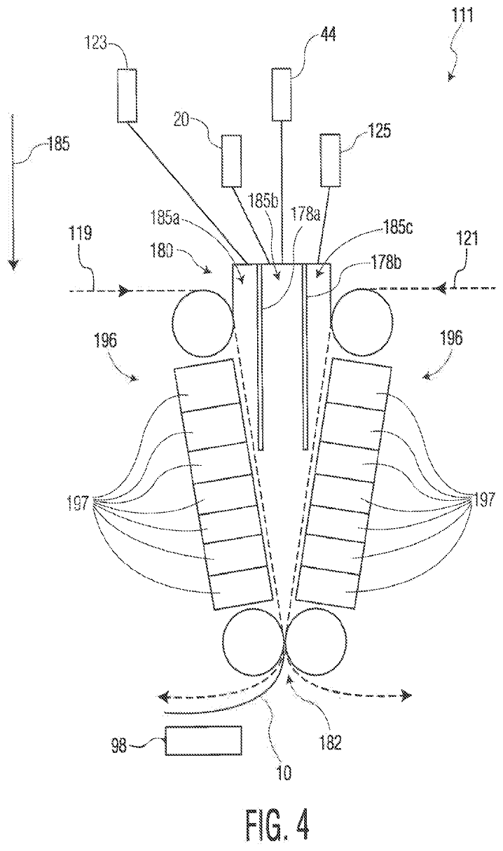

While the apparatus 11 and method described in FIG. 2 is one exemplary embodiment for forming a multi-layer substrate 10, an alternative embodiment of an apparatus 111 and method of forming an multi-layer substrate 10 is depicted in FIG. 4. The apparatus 111 of FIG. 4 can be used as part of a similar foam forming process as described above with respect to FIG. 2, however, the headbox 180 is a vertical twin former as is known in the art. The headbox 180 can include first and second foraminous elements 119, 121. The first and second foraminous elements 119, 121 can help define an interior volume of the headbox 180. The headbox 180 can include a first divider 178a and a second divider 178b that can provide first, second, and third z-directional layers 185a, 185b, 185c within the headbox 180 similar to the discussion above with the headbox 80 in FIG. 3, but the layers 185a, 185b, 185c in FIG. 4 are in a vertical orientation with respect to one another due to the vertical orientation of the headbox 180. The apparatus 111 can include a dewatering system 196 that can include a series of vacuum elements 197 disposed adjacent each foraminous element 119, 121. In some embodiments, a first supply of fibers 20 can be supplied to the headbox 180, and in some embodiments, the first supply of fibers 20 can be in a foam. The supply of the fibers 20 can include at least some absorbent fibers. The supply of the component 44 can also be supplied directly to the headbox 180, and in some embodiments, the supply of the component 44 may be in a foam. The supply of the fibers 20 and component 44 can be delivered to the second z-directional layer 185b of the headbox 180. It is to be noted that in some embodiments, the second z-directional layer 185b of the headbox 180 may only be provided with the supply of the component 44 and not a supply of fibers 20. In some embodiments, a second supply of fibers 123 can be provided to the headbox 180, and in some embodiments, can be in a foam. The second supply of fibers 123 can be provided to the first z-directional layer 185a of the headbox 180. In some embodiments, a third supply of fibers 125 can be provided to the headbox 180, and in some embodiments, can be in a foam. The third supply of fibers 125 can be provided to the third z-directional layer 185c of the headbox 180. The fibers 20, 123, 125 and component 44 can be processed through the headbox 180 in a machine direction 185 towards the outlet 182 of the headbox 180 to provide an absorbent material 10, similar to the apparatus 11 described in FIG. 2.

The apparatuses 11, 111 as described herein can also include a drying system 98 to further dry and/or cure the absorbent material 10, 110. The drying system 98 can apply heat to the absorbent material 10, such as by providing heated air in a through-air drying system.

In some embodiments, the apparatus 11, 111 can include a winding system 99 (as shown in FIG. 2) that can be configured to wind the absorbent material 10, 110 in a roll fashion. In other embodiments, the apparatus 11, 111 can festoon the absorbent material 10, 110 or collect the absorbent material 10, 110 in any other suitable configuration, such as spooling.

As described above, the foam forming processes as described herein can include a foaming fluid. In some embodiments, the foaming fluid can comprise between about 85% to about 99.99% of the foam (by weight). In some embodiments, the foaming fluid used to make the foam can comprise at east about 85% of the foam (by weight). In certain embodiments, the foaming fluid can comprise between about 90% and about 99.9% % of the foam (by weight). In certain other embodiments, the foaming fluid can comprise between about 93% and 99.5% of the foam or even between about 95% and about 99.0% of the foam (by weight). In preferred embodiments, the foaming fluid can be water, however, it is contemplated that other processes may utilize other foaming fluids.

The foam forming processes as described herein can utilize one or more surfactants. The fibers and surfactant, together with the foaming liquid and any additional components, can form a stable dispersion capable of substantially retaining a high degree of porosity for longer than the drying process. In this regard, the surfactant is selected so as to provide a foam having a foam half life of at least 2 minutes, more desirably at least 5 minutes, and most desirably at least 10 minutes. A foam half life can be a function of surfactant types, surfactant concentrations, foam compositions/solid level and mixing power/air content in a foam. The foaming surfactant used in the foam can be selected from one or more known in the art that are capable of providing the desired degree of foam stability. In this regard, the foaming surfactant can be selected from anionic, cationic, nonionic and amphoteric surfactants provided they, alone or in combination with other components, provide the necessary foam stability, or foam half life. As will be appreciated, more than one surfactant can be used, including different types of surfactants, as long as they are compatible, and more than one surfactant of the same type. For example, a combination of a cationic surfactant and a nonionic surfactant or a combination of an anionic surfactant and a nonionic surfactant may be used in some embodiments due to their compatibilities. However, in some embodiments, a combination of a cationic surfactant and an anionic surfactant may not be satisfactory to combine due to incompatibilities between the surfactants.

Anionic surfactants believed suitable for use with the present disclosure include, without limitation, anionic sulfate surfactants, alkyl ether sulfonates, alkylaryl sulfonates, or mixtures or combinations thereof. Examples of alkylaryl sulfonates include, without limitation, alkyl benzene sulfonic acids and their salts, dialkylbenzene disulfonic acids and their salts, dialkylbenzene sulfonic acids and their salts, alkylphenol sulfonic acids/condensed alkylphenol sulfonic acids and their salts, or mixture or combinations thereof. Examples of additional anionic surfactants believed suitable for use in the present disclosure include alkali metal sulforicinates, sulfonated glyceryl esters of fatty acids such as sulfonated monoglycerides of coconut oil acids, salts of sulfonated monovalent alcohol esters such as sodium oleylisethianate, metal soaps of fatty acids, amides of amino sulfonic acids such as the sodium salt of oleyl methyl tauride, sulfonated products of fatty acids nitriles such as palmitonitrile sulfonate, alkali metal alkyl sulfates such as sodium lauryl sulfate, ammonium lauryl sulfate or triethanolamine lauryl sulfate, ether sulfates having alkyl groups of 8 or more carbon atoms such as sodium lauryl ether sulfate, ammonium lauryl ether sulfate, sodium alkyl aryl ether sulfates, and ammonium alkyl aryl ether sulfates, sulphuric esters of polyoxyethylene alkyl ether, sodium salts, potassium salts, and amine salts of alkylnapthylsulfonic acid. Certain phosphate surfactants including phosphate esters such as sodium lauryl phosphate esters or those available from the Dow Chemical Company under the tradename TRITON are also believed suitable for use herewith. A particularly desired anionic surfactant is sodium dodecyl sulfate (SDS).

Cationic surfactants are also believed suitable for use with the present disclosure for manufacturing some embodiments of substrates. In some embodiments, such as those including superabsorbent material, cationic surfactants may be less preferable to use due to potential interaction between the cationic surfactant(s) and the superabsorbent material, which may be anionic. Foaming cationic surfactants include, without limitation, monocarbyl ammonium salts, dicarbyl ammonium salts, tricarbyl ammonium salts, monocarbyl phosphonium salts, dicarbyl phosphonium salts, tricarbyl phosphonium salts, carbylcarboxy salts, quaternary ammonium salts, imidazolines, ethoxylated amines, quaternary phospholipids and so forth. Examples of additional cationic surfactants include various fatty acid amines and amides and their derivatives, and the salts of the fatty acid amines and amides. Examples of aliphatic fatty acid amines include dodecylamine acetate, octadecylamine acetate, and acetates of the amines of tallow fatty acids, homologues of aromatic amines having fatty acids such as dodecylanalin, fatty amides derived from aliphatic diamines such as undecylimidazoline, fatty amides derived from aliphatic diamines such as undecylimidazoline, fatty amides derived from disubstituted amines such as oleylaminodiethylamine, derivatives of ethylene diamine, quaternary ammonium compounds and their salts which are exemplified by tallow trimethyl ammonium chloride, dioctadecyldimethyl ammonium chloride, didodecyldimethyl ammonium chloride, dihexadecyl ammonium chloride, alkyltrimethylammonium hydroxides, dioctadecyldimethylammonium hydroxide, tallow trimethylammonium hydroxide, trimethylammonium hydroxide, methylpolyoxyethylene cocoammonium chloride, and dipalmityl hydroxyethylammonium methosulfate, amide derivatives of amino alcohols such as beta-hydroxylethylstearylamide, and amine salts of long chain fatty acids. Further examples of cationic surfactants believed suitable for use with the present disclosure include benzalkonium chloride, benzethonium chloride, cetrimonium bromide, distearyldimethylammonium chloride, tetramethylammonium hydroxide, and so forth.

Nonionic surfactants believed suitable for use in the present disclosure include, without limitation, condensates of ethylene oxide with a long chain fatty alcohol or fatty acid, condensates of ethylene oxide with an amine or an amide, condensation products of ethylene and propylene oxides, fatty acid alkylol amide and fatty amine oxides. Various additional examples of non-ionic surfactants include stearyl alcohol, sorbitan monostearate, octyl glucoside, octaethylene glycol monododecyl ether, lauryl glucoside, cetyl alcohol, cocamide MEA, monolaurin, polyoxyalkylene alkyl ethers such as polyethylene glycol long chain (12-14C) alkyl ether, polyoxyalkylene sorbitan ethers, polyoxyalkylene alkoxylate esters, polyoxyalkylene alkylphenol ethers, ethylene glycol propylene glycol copolymers, polyvinyl alcohol, alkylpolysaccharides, polyethylene glycol sorbitan monooleate, octylphenol ethylene oxide, and so forth. Non-ionic surfactants may be preferable when foam forming absorbent materials 10, 110 with SAM. If there is residual ionic surfactant, the increase in ionic strength in the insult can reduce SAM swelling for use of the absorbent materials 10, 110 in personal care absorbent articles.

The foaming surfactant can be used in varying amounts as necessary to achieve the desired foam stability and air-content in the foam. In certain embodiments, the foaming surfactant can comprise between about 0.005% and about 5% of the foam (by weight). In certain embodiments the foaming surfactant can comprise between about 0.05% and about 3% of the foam or even between about 0.05% and about 2% of the foam (by weight).

As noted above, the apparatus 11, 111 and methods described herein can include providing a fibers from a supply of fibers 20, 37, 70, 123, 125. In some embodiments, the fibers can be suspending in a fluid supply 16, 28, 33, 68 that can be a foam. The foam suspension of fibers can provide one or more supply of fibers. As described above, fibers utilized herein can include natural fibers and/or synthetic fibers. In some embodiments, a fiber supply 20, 37, 70, 123, 125 can include only natural fibers or only synthetic fibers. In other embodiments, a fiber supply 20, 37, 70, 123, 125 can include a mixture of natural fibers and synthetic fibers. Some fibers being utilized herein can be absorbent, whereas other fibers utilized herein can be non-absorbent. Non-absorbent fibers can provide features for the substrates that are formed from the methods and apparatuses described herein, such as improved intake or distribution of fluids.

In some embodiments, a fluid supply 16, 28, 33, 68 can include binder materials (as described above) that can be provided along with or independent of the supply of the fibers 20, 37, 70, 123, 125 or the supply of the component 44.

Binders can additionally or alternatively be provided in a liquid form, such as latex emulsions. and can comprise between about 0% and about 10% of the foam (by weight). In certain embodiments the non-fibrous binder can comprise between about 0.1% and 10% of the foam (by weight) or even between about 0.2% and about 5% or even between about 0.5% and about 2% of the foam (by weight).

Binder fibers, when used, may be added proportionally to the other components to achieve the desired fiber ratios and structure while maintaining the total solids content of the foam below the amounts stated above. As an example, in some embodiments, binder fibers can comprise between about 0% and about 50% of the total fiber weight, and more preferably, between about 5% to about 40% of the total fiber weight in some embodiments.

In some embodiments, if a fluid supply 16, 28, 33, 68 is configured as a foam the foam may optionally also include one or more foam stabilizers known in the art and that are compatible with the components of the foam and further do not interfere with the hydrogen bonding as between the cellulosic fibers. Foam stabilizing agents believed suitable for use in the present disclosure, without limitation, one or more zwitterionic compounds, amine oxides, alkylated polyalkylene oxides, or mixture or combinations thereof. Specific examples of foam stabilizers includes, without limitation, cocoamine oxide, isononyldimethylamine oxide, n-dodecyldimethylamine oxide, and so forth.

In some embodiments, if utilized, the foam stabilizer can comprise between about 0.01% and about 2% of the foam (by weight). In certain embodiments, the foam stabilizer can comprise between about 0.05% and 1% of the foam or even between about 0.1 and about 0.5% of the foam (by weight). As mentioned above, foam forming processes can include adding one or more components 44 as additional additives that will be incorporated into the absorbent material 10, 110, such as SAM. In some embodiments incorporating SAM, the SAM can comprise between about 0% and about 40% of the foam (by weight). In certain embodiments, SAM can comprise between about 1% and about 30% of the foam (by weight) or even between about 10% and about 30% of the foam (by weight).

If used, wet and dry strength additives can comprise between about 0.01 and about 5% of the dry weight of cellulose fibers. In certain embodiments, the strength additives can comprise between about 0.05% and about 2% of the dry weight of cellulose fibers or even between about 0.1% and about 1% of the dry weight of cellulose fibers.

When employed, miscellaneous components that may also be used in the absorbent material (as described above, such as, pigments, anti-microbial agents, etc.) can desirably comprise less than about 2% of the foam (by weight) and still more desirably less than about 1% of the foam (by weight) and even less than about 0.5% of the foam (by weight).

In some embodiments, the solids content, including the fibers or particulates contained herein, desirably comprise no more than about 40% of the foam. In certain embodiments the cellulosic fibers can comprise between about 0.1% and about 5% of the foam or between about 0.2 and about 4% of the foam or even between about 0.5% and about 2% of the foam.

The methods and apparatuses 11, 111 as described herein can be beneficial for forming one or more absorbent materials 10, 110. The absorbent materials 10, 110 as described herein can be useful as components of personal care products. For example, in one embodiment, the absorbent material 10, 110 as described herein can be an absorbent composite for personal care absorbent articles. The multi-layer absorbent materials 10, 110 as described herein may also be beneficial for using in other products, such as, but not limited to facial tissues, bath tissues, wipes, and wipers.

The multi-layer substrate made in accordance with the present disclosure can be incorporated into all different types of absorbent articles. The absorbent article, for instance, can be a diaper, a child training pant, or other child absorbent pant. The absorbent article can also be an adult incontinence product. The absorbent article can include a fluid permeable liner and an outer cover. The multi-layer substrate of the present disclosure can form an absorbent core positioned between the fluid permeable liner and the outer cover. In one embodiment, a surge layer can be placed in between the absorbent core and the fluid permeable liner for directing and channeling fluids into the absorbent core in a fast and efficient manner.

The present disclosure may be better understood with reference to the following examples.

### Test Procedures

### Tensile Properties:

The strip tensile strength values were determined in substantial accordance with ASTM Standard D-5034. Specifically, a sample was cut or otherwise provided with size dimensions that measured 3 inches (76.2 millimeters) (width)×6 inches (152.4 millimeters) (length). A constant-rate-of-extension type of tensile tester was employed. The tensile testing system was a Sintech Tensile Tester, which is available from MTS Corp. of Eden Prairie, Minn. The tensile tester was equipped with TESTWORKS 4.08B software from MTS Corporation to support the testing. An appropriate load cell was selected so that the tested value fell within the range of 10-90% of the full scale load. The sample was held between grips having a front and back face measuring 1 inch (25.4 milimeters)×3 inches (76 millimeters). The grip faces were rubberized, and the longer dimension of the grip was perpendicular to the direction of pull. The tensile test was run at a 300 mm per minute rate with a gauge length of 3 inches and a break sensitivity of 40%. Three samples were tested along the machine-direction (“MD”) and three samples were tested by along the cross direction (“CD”). The ultimate tensile strength (“peak load”) was recorded in grams force. The result can be normalized by dividing by the three inch width. Test specimens can be tested at widths from 1 inch to 5 inches and normalized.

### Absorbency Under Load (AUL) Test:

Specimen Preparation: Cut absorbent composite specimens with a 15/16″ circular die and weight sort the specimens to get N=5 that are closest to the target basis weight of interest. If the absorbent composite specimens do not have a homogenous distribution of absorbent materials, mark which surface has the highest percentage of superabsorbent material closest to the surface.

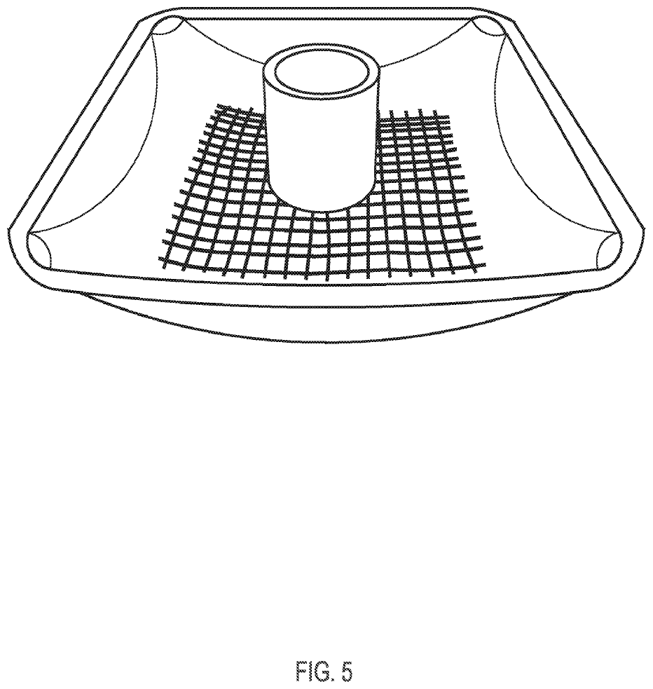

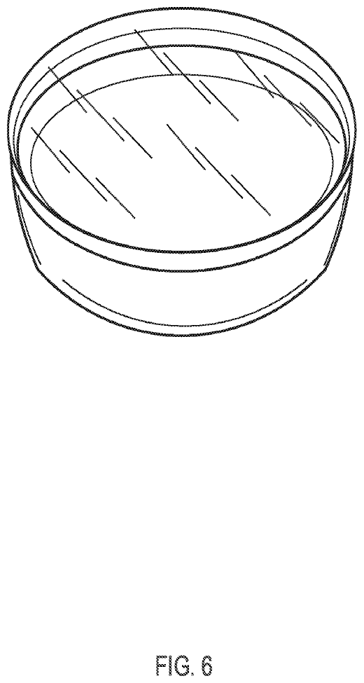

FIGS. 5 and 6 illustrate the equipment used during the test including the AUL cylinder (acrylic) having a 15/16″ ID and the plastic platen weight.

AUL Test Procedure: Place the 15/16″ diameter absorbent composite specimen into the AUL cylinder. Use a lab spatula to gently tap/push the side edges of the absorbent composite specimen down to fit snuggly in the bottom of the AUL cylinder. If the absorbent composite specimens do not have a homogenous distribution of SAM, place the specimen surface that has the highest percentage of SAM towards the bottom of the AUL cylinder.

Gently place the plastic platen (0.01 PSI) weight (acrylic) into the AUL cylinder (see FIG. 6).

Weigh the AUL Cylinder+Specimen+Platen unit and record the weight to the nearest milligram as the Dry AUL Cylinder+Specimen+Platen unit weight.

Pour a small amount of test fluid (0.9% NaCl saline) in the fluid bath with the screen on the bottom as shown in FIG. 5. The screen can be stainless steel or a plastic mesh screen with open areas. Pour enough fluid so that the level is just above the screen.

Simultaneously start the timer and place the AUL Cylinder+Specimen+Platen unit onto the screen in the fluid bath. If necessary, add more test fluid. The fluid level in the bath should be at a height to provide at least a 1 cm positive head above the base of the cylinder.
Note: Five specimens at each predetermined time interval must be tested. Required time intervals are 30 seconds and 60 minutes.

Remove the cylinder from the fluid bath at the designated time and wipe the exterior of the cylinder with paper toweling or tissue.

Weigh the AUL Cylinder+Specimen+Platen unit and record the weight to the nearest milligram as the Wet AUL Cylinder+Specimen+Platen unit weight.

Repeat for all test specimens of interest.

Subtract the average Dry AUL Cylinder+Specimen+Platen unit weight from the average Wet AUL Cylinder+Specimen+Platen unit weight to derive the average total AUL.

### Cradle Test Method:

The Cradle Test replicates real-life positioning of a garment on a wearer, and can be used to determine intake rates, flowback, and fluid distribution of a garment. This method uses a slotted cradle, as shown in FIGS. 4b and of U.S. Pat. No. 6,727,404, both made up of a water-resistant material such as acrylic plastic and simulating body curvature of a wearer. Two different cradle sizes were used-one for adult care garments and one for baby diapers.

The adult cradle has an overall length of 425 mm, a side-to-side width of 425 mm and a height of 290 mm (including about 50 mm height below the slot). Material used in the construction varies in thickness from 6 mm to 12 mm. The cradle has a 6 mm wide slot at the lowest point which runs the length of the cradle. The curvature of the cradle is formed by a 75-degree angle.

The baby cradle has an overall length of 305 mm, a side-to-side width of 350 mm in the slot direction and a height of 255 mm (including 57 mm height below the slot). Material used in the construction varies in thickness from 6 mm to 12 mm. The cradle has a 6 mm wide slot at the lowest point which runs the length of the cradle. The curvature of the cradle is formed by a 60-degree angle.

### 1. Product Preparation

A. For adult care garments, cut open the three-dimensional pant style products at the sides or side seams to make the product two-dimensional.
B. Do not snip the leg and containment flap elastics.
C. Weigh the product to the nearest 0.01 gram value and record the value. D. Measure the pad length (using a light board) to the nearest 1 mm value and record the value.
E. Measure the product length and mark the center.
F. Mark the insult area at 95 mm forward from the center of the product to the nearest 1 mm value. Center the measurement in the cross direction.

### 2. Product Testing (Test Fluid=0.9 w/v % Saline Solution)

A. Verify that the pump delivers the required test fluid amount for the insult+/−0.5 ml. The flow rate should be set at 8 ml/second. Test fluid amount will be 50 ml for adult care products or 85 ml for baby diaper products. The hose end or nozzle should have an exit diameter of 0.125 inches.
B. For the slotted cradle, place a capture container of known weight under the cradle slot to catch the fluid overflow. Measure the weight of the capture container to the nearest 0.01 grams. Note: low capacity products without flaps (i.e., cloth underwear, cloth training pants, vinyl/cloth training pants) will be prone to overflow.
D. Position the specimen, liner/inside side up, with the “pre-marked” center of the product lined up with and touching the lowest point in the cradle. The entire length of the outer cover/outside of the product should make contact with the cradle. Clip or otherwise attach the product to the cradle at the front and back waist edges to keep it in place. Gently pull on the front/back waist of the specimen to smooth out any wrinkles or creases in the product. For the cradle testing, all product codes will be insulted at a point 95 mm forward from the center of the product.
E. Hold the nozzle above the target area and perpendicular to the specimen. The bottom of the nozzle should be within 5 to 10 mm from the specimen.
F. Initiate the insult and start the stopwatch when the testing fluid leaves the nozzle. As soon as the insult is complete, move the nozzle aside to observe the testing fluid.
G. Stop the stopwatch immediately when the testing fluid is not visible on the specimen surface. Record the intake time to the nearest 0.01 second. If the fluid overflows into the capture container, the intake time will be recorded when no more fluid is visible on the surface.
H. Once the insult is absorbed, immediately set the timer for 15 mins. Leave the specimen in the cradle for the entire wait time.
I. Repeat steps F, G, and H two more times for a total of 3 insults separated by 15 minutes.

### Fluid Intake Under Pressure (FIUP) Test Method:

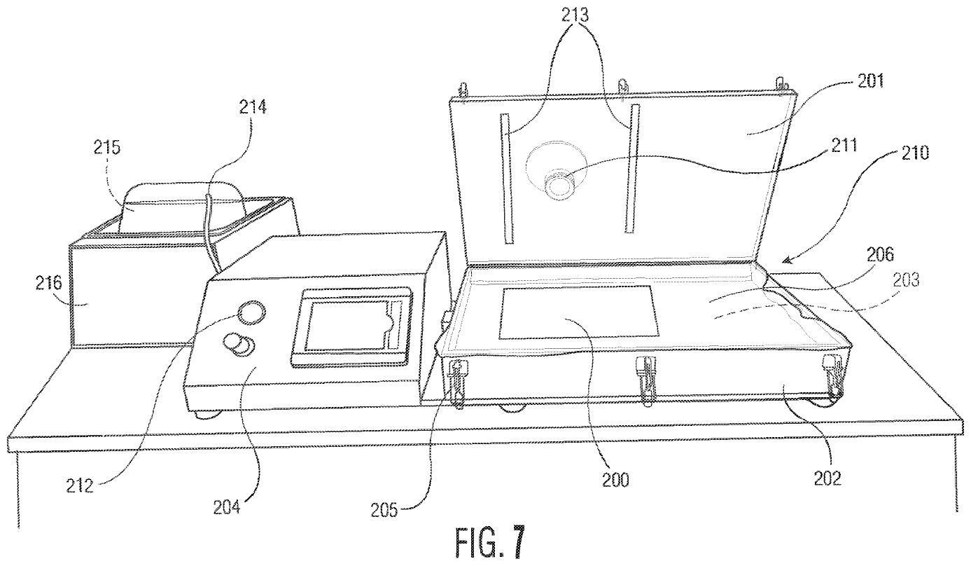

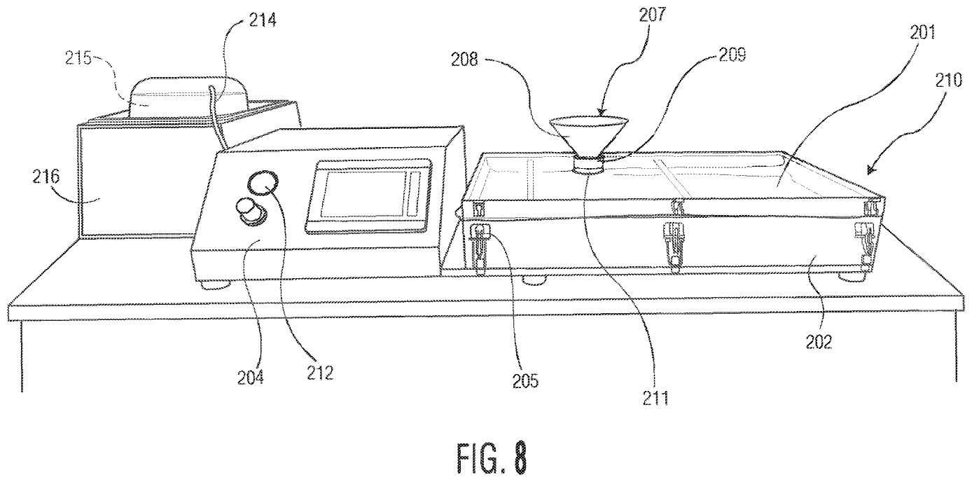

The first, second, third, and fourth intake times of experimental codes were measured according to the following protocol and by the exemplary equipment illustrated in FIG. 7 for a Fluid Intake Under Pressure (FIUP) Test. The specimens 200 were prepared to the following dimensions: 295 mm in length and 70 mm in width and placed between the topsheet with flaps and the back sheet from the commercially available Poise® Ultra Thin 5-drop pads. The topsheet can be a 20 gsm polypropylene spunbond nonwoven liner material with hydrophilic treatment, such as XHBY21520 MYSQS215 material provided by Lanxi Xinghan Plastic Material Co. (Hengyao). The back sheet can be a 24 gsm polyethylene film. For specimens without an intake layer, a fresh piece of 185 mm by 49 mm intake layer material of 42 gsm polyethylene/polypropylene bicomponent TABCW (Jing Lan) was placed over the core to serve as an intake layer and had 6 gsm of adhesive applied (from a swirl of adhesive on release paper) to the top and bottom of the intake layer. The sides of the sample 200 are sealed with double-sided tape. The samples 200 were brought to TAPPI conditions for at least 4 hours. The FIUP Test uses a “bladder box” 210 as illustrated in FIGS. 7 and 8. The bladder box 210 includes a cover 201, a housing 202, an inflatable bladder 203, and a control unit 204. The cover 201 can be made from a clear material, such as clear, cast acrylic. The cover 201 can be hinged to the housing 202. The housing 202 can be constructed from aluminum can be of the size of 62 cm×40 cm×15 cm. The housing 202 can also include latches 205, such as the three latches 205 depicted in FIGS. 7 and 8, for securing the cover 201 to the housing 202. When the cover 201 is opened, the test specimen 200 can be laid on top of a thin plastic film 206 laid on top of the bladder 203. The test specimen should be laid on the film 206 and bladder 203 such that the specimen 200 is centered under the intake port 207. The bladder 203 can be an inflatable bladder, such as an Aero Tec Labs bladder, that can fit within the housing 202 and that can be filled with compressed air.

The intake port 207 can include a threaded funnel 208 that threads into a threaded plug 209 having a 1″ diameter opening at the bottom of the threaded plug 209 and provides for communication to the test specimen 200. The intake port 207 can also include an O-ring 211 that seals the threaded plug 209 with the cover 201. The intake port 207 can also include a round, flat gasket (not shown) to seal between the threaded funnel 208 and the threaded plug 209. The bottom of the intake port 207 should be flush with the underside of the cover 201.

The control unit 204 can be a process controller such as 1/16 DIN Fuzzy Logic; Example: Omega, part number CN48001-F1-AL2: G1, or equivalent, and can be configured to be in communication with a pressure transmitter measuring the pressure of the bladder 203. An exemplary pressure transmitter can be an Omega Engineering, part number PX181-015GSV. The control unit 204 can also be in communication with a fluid dispensing pump (e.g., Cole-Parmer peristaltic pump, P/N 07551-20) and pump head (P/N 77201-60) that is set up to deliver fluid to the test sample at a specified flow rate of 8 mL/s via clear pump tubing 214 (e.g., Masterflex clear tubing L/S 14, L/S 25, or L/S 17). The end fitting on the tubing can have an exit diameter of 0.125″, such as Cole-Parmer Reducing Connector, Nylon, ¼″× 3/16″, Item No. 30622-30.

After the test specimen 200 is set in the bladder box housing 202 by being centered below the intake port 207. As illustrated in FIG. 7, the bottom of the cover 201 can include two strips of hook tape 213 (e.g., Item #1055, Dariss Brand) that are used to help secure the test specimen 200. After the sample is centered, the cover 201 is closed and latches 205 are latched. The hook tape 213 should be applied to the cover 201 such that the hook tape 213 only touches non-absorbent material of the test specimen 200. The power for the control unit 204 is turned on to set the bladder 203 pressure to 0.25 psi. Once the control unit 204 identifies that the bladder 203 has reached a stable pressure of 0.25 psi, a pressure gauge 212 can be checked to verify that the pressure in the bladder 203 is within 0.25+/−0.01 psi. If the pressure is not within 0.01 psi of 0.25 psi, the test should be stopped and the set pressure should be adjusted to compensate until the pressure gauge 212 reads within 0.01 psi of 25 psi.

The insult liquid used for the FIUP test is 0.9±0.005% (w/w) aqueous isotonic saline 215 that is placed in a heated water bath 216 at a temperature of 98.6±1.8 °F/37±1 °C prior to testing. The saline solution 215 temperature should be confirmed with a thermometer prior to insulting the test specimen 200. The first insult is a 25 mL insult and is supplied through the intake port 207 by aiming the fluid at the bottom angled side of the funnel 208. The first intake time of the first insult begins once the pump is turned on to deliver fluid to the intake port 207 and continues until all droplets of fluid have been absorbed within top layer of the test specimen 200. The second 25 mL insult is applied 15 minutes after the first insult is fully absorbed and the second intake time is measured in the same manner as the first insult time. The third 25 mL insult is applied 15 minutes after the second insult is fully absorbed and the third intake time is measured in the same manner as described above. The fourth 25 mL insult is applied 15 minutes after the third insult is fully absorbed and the fourth intake time is measured in the same manner as described above.

After the fourth intake time is recorded, a timer should be started to allow two minutes to pass. The control unit 204 is then calibrated to stop the test by releasing the bladder 203 pressure in the bladder box 210. If any point during the insult testing there is any fluid runoff beyond the test specimen 200 on to the plastic sheet 206 covering the bladder 203, the test should be marked as a “FAIL” and not recorded.

The testing is conducted with a sample set of N=5.

### Rewet Test Method:

The rewet for the experimental codes was measured by using the same specimen from the FIUP test discussed above. The rewet test is a continued test after the FIUP test was completed. Specifically, 2 minutes after the fourth insult of the FIUP test is complete, the sample is removed from the bladder box 210 and placed onto a flat surface, insult side facing up. The test is completed using two stacked pieces of blotting paper (e.g., 300 g/m² (100 lb./ream)-Verigood Grade 88 by 300±13 mm (3.5 by 12±0.5 inch) to absorb the free saline from the insulting point of the specimen 200 under an external load after the FIUP test. The two pieces of blotting paper were pre-weighed and each had a dimension of 3.5″×12″ and would be placed to cover the center of the specimen's insulting point by removing the FIUP testing board and adding a cylindrical weight of 249 g and having a 1 inch diameter on the top of the blotting papers at the insult point to create a pressure of 0.7 psi for a period of two minutes. The mass of the wetted blotter papers is then measured and the rewet is calculated as: Rewet=total wet mass-dry mass. The higher the amount of wet weight measured from the test, the higher the rewet value the specimen had.

### Thickness Measurement Method:

Both dry thickness and wet thickness measurements of experimental codes were measured as part of the FIUP test discussed above. The thickness measurements utilize a standard bulk tester with clear, cast acrylic foot that provides 0.05 psi. The dry thickness measures the dry bulk at the center point when the sample is dry and measures the thickness of the sample in a full product form as placed in a Poise® Ultrathin chassis that includes flaps, outer cover, and liner (only the outer cover and liner form part of the thickness measurement, as the flaps are outside the platen area). The wet thickness is measured after the rewet testing is complete by measuring the bulk at the center point.

### Example No. 1

In the examples below, various different multi-layer substrates were produced and tested for strength and fluid control properties. All of the samples below were produced using a foam-forming process. The following multi-layer substrates were produced:

| Sample | | | |
|---|---|---|---|
| No. | First layer | Second layer | Third layer |
| 1 | 50 gsm fiber (35% | 400 gsm SAM, 75 gsm fiber (30% | None |
| | bicomponent binder | bicomponent binder fibers, 30% PET | |
| | fibers, 65% PET fibers) | fibers, 40% crosslinked pulp fiber) | |
| 2 | 50 gsm fiber (35% | 400 gsm SAM, 75 gsm fiber (30% | None |
| | bicomponent binder | bicomponent binder fibers, 30% PET | |
| | fibers, 65% PET fibers) | fibers, 40% crosslinked pulp fiber) | |
| 3 | 40 gsm fiber (35% | 345 gsm SAM, 20 gsm fiber (40% | None |
| | bicomponent binder | bicomponent binder fibers, 10% PET | |
| | fibers, 65% PET fibers) | fibers, 25% crosslinked pulp fiber, 25% | |
| | | softwood pulp fiber) | |
| 4 | 40 gsm fiber (35% | 400 gsm SAM, 20 gsm fiber (3% | 20 gsm fiber (3% |
| | bicomponent binder | bicomponent binder fibers, 30% PET | bicomponent binder |
| | fibers, 65% PET fibers) | fibers, 67% crosslinked pulp fiber) | fibers, 30% PET fibers, |
| | | | 67% crosslinked pulp |
| | | | fiber) |
| 5 | 15 gsm fiber (30% | 380 gsm SAM, 95 gsm fiber (0% | 20 gsm fiber (75% |
| | bicomponent binder | bicomponent binder fibers, 30% PET | bicomponent binder |
| | fibers, 40% PET fibers, | fibers, 70% crosslinked pulp fiber) | fibers, 25% softwood pulp |
| | 30% crosslinked pulp | | fiber) |
| | fiber) | | |
| 6 | 50 gsm fiber (35% | 415 gsm SAM, 90 gsm fiber (5% | None |
| | bicomponent binder | bicomponent binder fibers, 35% PET | |
| | fibers, 65% PET fibers) | fibers, 60% crosslinked pulp fiber) | |
| 7 | 50 gsm fiber (35% | 415 gsm SAM, 120 gsm fiber (5% | None |
| | bicomponent binder | bicomponent binder fibers, 35% PET | |
| | fibers, 65% PET fibers) | fibers, 60% crosslinked pulp fiber) | |
| 8 | 15 gsm fiber (30% | 415 gsm SAM, 90 gsm fiber (10% | None |
| | bicomponent binder | bicomponent binder fibers, 30% PET | |
| | fibers, 40% PET fibers, | fibers, 60% crosslinked pulp fiber) | |
| | 30% crosslinked pulp | | |
| | fiber) | | |
| 9 | 15 gsm fiber (30% | 350 gsm SAM, 95 gsm fiber (10% | 25 gsm fiber (30% |
| | bicomponent binder | bicomponent binder fibers, 30% PET | bicomponent binder |
| | fibers, 40% PET fibers, | fibers, 60% crosslinked pulp fiber) | fibers, 40% PET fibers, |
| | 30% crosslinked pulp | | 30% crosslinked pulp |
| | fiber) | | fiber) |
| 10 | 15 gsm fiber (30% | 380 gsm SAM, 95 gsm fiber (0% | 25 gsm fiber (30% |
| | bicomponent binder | bicomponent binder fibers, 30% PET | bicomponent binder |
| | fibers, 40% PET fibers, | fibers, 70% crosslinked pulp fiber) | fibers, 40% PET fibers, |
| | 30% crosslinked pulp | | 30% crosslinked pulp |
| | fiber) | | fiber) |
| 11 | 15 gsm fiber (30% | 350 gsm SAM, 95 gsm fiber (10% | 25 gsm fiber (30% |
| | bicomponent binder | bicomponent binder fibers, 30% PET | bicomponent binder |
| | fibers, 40% PET fibers, | fibers, 60% crosslinked pulp fiber) | fibers, 40% PET fibers, |
| | 30% crosslinked pulp | | 30% crosslinked pulp |
| | fiber) | | fiber) |
| 12 | 50 gsm fiber (35% | 415 gsm SAM, 20 gsm fiber (40% | None |
| | bicomponent binder | bicomponent binder fibers, 30% PET | |
| | fibers, 65% PET fibers) | fibers, 30% crosslinked pulp fiber) | |
| 13 | 50 gsm fiber (35% FV | 400 gsm SAM, 50 gsm fiber (30% PET | 25 gsm fiber (35% FV |
| | ESC222CTWL | fibers, 70% crosslinked pulp fiber) | ESC222CTWL |
| | bicomponent binder | | bicomponent binder |
| | fibers, 65% Trevira T-245 | | fibers, 65% Trevira T-245 |
| | PET 6.7dTex 6 mm) | | PET 6.7dTex 6 mm) |
| 14 | 50 gsm fiber (35% FV | 320 gsm SAM, 50 gsm fiber (30% PET | 25 gsm fiber (35% FV |
| | ESC222CTWL | fibers, 70% crosslinked pulp fiber) | ESC222CTWL |
| | bicomponent binder | | bicomponent binder |
| | fibers, 65% Trevira T-245 | | fibers, 65% Trevira T-245 |
| | PET 6.7dTex 6 mm) | | PET 6.7dTex 6 mm) |

The superabsorbent material (SAM) was commercially available SXM 5660 manufactured by Evonik, except Sample Nos. 4, 13 and 14 contained product code LK601N obtained from LG and Sample No. 1 contained product code 9807X obtained from BASF. The PET fibers used had a length of 6 mm, were crimped, and had a size of 6.7 dTex. The binder fibers used were a polyethylene/PET sheath/core structure that had a 2.2 dTex fiber diameter and a 6 mm fiber length, manufactured by Trevira. The crosslinked pulp fiber used was obtained from International Paper.

In this example, Sample Nos. 1-14 were tested for tensile strength and tested according to the AUL absorbency test. The following results were obtained:

| Sample | Tensile Peak | .01 psi AUL | .01 psi AUL |
|---|---|---|---|
| No. | Load (gf/3 in) | G/G 30 sec | G/G 60 min |
| 1 | 3414 | 6.1 | 20.5 |
| 2 | 3646 | 9.3 | 23.2 |
| 3 | 2133 | 7 | 24.7 |
| 4 | 2089 | 16 | 35.2 |
| 5 | 3340 | 12.7 | 33 |
| 6 | 1390 | 9.5 | 25.5 |
| 7 | 1651 | 11 | 24.3 |
| 8 | 1195 | 12.6 | 25.1 |
| 9 | 3394 | 11.7 | 27.9 |
| 10 | 997 | 14.53 | 31.3 |
| 11 | 2227 | 10.29 | 33.3 |
| 12 | 1498 | 7 | 21.6 |
| 13 | 7655 | 16.7 | 31.4 |
| 14 | 4805 | 17.6 | 32.1 |

As shown above, multi-layer substrates made in accordance with the present disclosure that do not contain substantial amounts of binder fibers in the second layer displayed higher 30 second AUL test results in combination with excellent strength properties.

### Example No. 2

Various substrates identified above in Example No. 1 were then tested for dry bulk, wet bulk, and according to the Cradle Test. A surge layer was placed over the top layer of the multi-layer substrate in some of the experiments. In the first set of experiments the material was tested in an adult care garment product, with 50 mL insult volume and absorbent dimensions of 75 mm×320 mm. In the second set of experiments the material was tested in a baby diaper product, with 85 mL insult volume and absorbent dimensions of 100 mm×354 mm.

The following results were obtained:

| Sample | Core dims. | | Dry bulk | Wet bulk | Cradle 1st | Cradle 2nd | Cradle 3rd |
|---|---|---|---|---|---|---|---|
| No. | (mm) | surge | (mm) | (mm) | intake (s) | intake (s) | intake (s) |
| 2 | 75 × 320 | none | na | na | 10.9 | 18.6 | 72.7 |
| 9 | 75 × 320 | 100 gsm | 4.8 | 15.1 | 8.7 | 9.0 | 19.7 |
| | | through air | | | | | |
| | | bonded and | | | | | |
| | | carded | | | | | |
| | | material | | | | | |
| 10 | 75 × 320 | 100 gsm | 5.2 | 16.5 | 8.0 | 7.6 | 9.3 |
| | | through air | | | | | |
| | | bonded and | | | | | |
| | | carded | | | | | |
| | | material | | | | | |

| Sample | | Dry bulk | Wet bulk | Cradle 1st | Cradle 2nd | Cradle 3rd |
|---|---|---|---|---|---|---|
| No. | surge | (mm) | (mm) | intake (s) | intake (s) | intake (s) |
| 5 | 55 gsm | 7.1 | 17.2 | 12.2 | 9.1 | 9.9 |
| | textured | | | | | |
| | hydroentangled | | | | | |
| 5 | 85 gsm through | 7.1 | 20.2 | 9.4 | 7.9 | 9.1 |
| | air bonded and | | | | | |
| | carded material | | | | | |
| 6 | none | 6.2 | 20.4 | 16.4 | 24.7 | 75.9 |
| 7 | none | 7.6 | 19.9 | 18.0 | 29.0 | 178.8 |
| 8 | 55 gsm | 8.0 | 19.6 | 11.1 | 16.2 | 192.2 |
| | textured | | | | | |
| | hydroentangled | | | | | |
| 9 | 55 gsm | 5.7 | 15.4 | 18.2 | 29.1 | 202 |
| | textured | | | | | |
| | hydroentangled | | | | | |
| 10 | 55 gsm | 5.2 | 18.1 | 14.3 | 9.7 | 24.9 |
| | textured | | | | | |
| | hydroentangled | | | | | |
| 12 | none | 6.1 | 17.4 | 43.5 | 115.5 | 294.1 |

As shown above, Sample Nos. 5 and 10 displayed excellent absorbency characteristics.

### Example No. 3

Various different multi-layer substrates were produced using a foam-forming process and tested for strength and fluid control properties. The following multi-layer substrates were produced:

| | | Top layer (Intake Layer) | | | | |
|---|---|---|---|---|---|---|
| | | | | Bicomponent | Bicomponent | |
| | | PET (13 | Bicomponent | Binder Fibers | Binder Fibers | |
| | Total | dtex- | Binder Fibers | (2.2 dtex-2 | (1.3 dtex-1.2 | Fiber |
| Sample | BW | 11.7 denier), | (6.7 dtex-6 | denier), | denier), | BW |
| No. | (gsm) | (6 mm) (%) | denier) (%) | (6 mm) (%) | (6 mm) (%) | (gsm) |
| 13 | 490 | 40 | 45 | 15 | — | 60 |
| 14 | 590 | 40 | 45 | 15 | — | 60 |
| 15 | 395 | 40 | 45 | 15 | — | 60 |
| 16 | 560 | 40 | 45 | 15 | — | 40 |
| 17 | 580 | 60 | 30 | 10 | — | 60 |
| 18 | 485 | 40 | 45 | 15 | — | 60 |
| 19 | 465 | 40 | 30 | — | 30 | 60 |
| 20 | 590 | 30 | 55 | 15 | — | 60 |
| 21 | 590 | 45 | 40 | 15 | — | 60 |
| 22 | 590 | 60 | 25 | 15 | — | 60 |

| | | Center layer | | | | | | |
|---|---|---|---|---|---|---|---|---|
| | | Bicomponent | | | | Bicomponent | | |
| | | Binder Fibers | PET (6.7 | | | Binder Fibers | | |
| | Total | (2.2 dtex-2 | dtex- | Crosslinked | SBSK | (1.3 dtex-1.2 | Fiber | |
| Sample | BW | denier), | 6 denier), | Pulp Fibers | (pulp) | denier, | BW | SAM |
| No. | (gsm) | (6 mm) (%) | (6 mm) (%) | (%) | (%) | (6 mm) (%) | (gsm) | (gsm) |
| 13 | 490 | 10 | 30 | 60 | — | — | 85 | 315 |
| 14 | 590 | 10 | 30 | 60 | — | — | 85 | 415 |
| 15 | 395 | 10 | 30 | 60 | — | — | 85 | 220 |
| 16 | 560 | 10 | 30 | 30 | 30 | — | 85 | 415 |
| 17 | 580 | 10 | 30 | 60 | — | — | 85 | 415 |
| 18 | 485 | 10 | 30 | 60 | — | — | 80 | 315 |
| 19 | 465 | — | — | 85 | — | 15 | 60 | 315 |
| 20 | 590 | 10 | 30 | 60 | — | — | 85 | 415 |
| 21 | 590 | 10 | 30 | 60 | — | — | 85 | 415 |
| 22 | 590 | 10 | 30 | 60 | — | — | 85 | 415 |

| | | Bottom layer | | | | |
|---|---|---|---|---|---|---|
| | | Bicomponent | | | Cross- | |
| | | Binder Fibers | | PET (0.6 | linked | |
| | Total | (1.3 dtex-1.2 | SBSK | dtex-0.5 | Pulp | Fiber |
| Sample | BW | denier), | (pulp) | denier), | Fibers | BW |
| No. | (gsm) | (6 mm) (%) | (%) | (12 mm) | (%) | (gsm) |
| 13 | 490 | 35 | 65 | — | — | 30 |
| 14 | 590 | 35 | 65 | — | — | 30 |
| 15 | 395 | 35 | 65 | — | — | 30 |
| 16 | 560 | 35 | 65 | — | — | 20 |
| 17 | 580 | 35 | 65 | — | — | 20 |
| 18 | 485 | 25 | — | 35 | 40 | 30 |
| 19 | 465 | 30 | 45 | 25 | — | 30 |
| 20 | 590 | 35 | 65 | — | — | 30 |
| 21 | 590 | 35 | 65 | — | — | 30 |
| 22 | 590 | 35 | 65 | — | — | 30 |

SBSK—Southern bleached softwood kraft fibers

The above multi-layer substrates were then tested for average tensile peak load (average of five samples) and absorbency under load (average of three samples). The following results were obtained:

| Sample | Average Tensile Peak Load | Average 0.01 psi |
|---|---|---|
| No. | (gf/3 in) | AUL (g/g) 30 sec |
| 13 | 5491 | 17.53 |
| 14 | 4577 | 16.67 |
| 15 | 6047 | 19.59 |
| 16 | 4218 | 16.49 |
| 17 | 4632 | 16.14 |
| 18 | 5693 | 15.79 |
| 19 | 10633 | 12.73 |
| 20 | 6996 | 15.55 |
| 21 | 6028 | 16.57 |
| 22 | 5549 | 16.49 |

As shown above, all of the multi-layer absorbent substrates displayed excellent tensile strength properties in combination with fluid handling properties.

All of the above substrates were also tested for Fluid Intake Under Pressure (FIUP) (38 ml/insult, total of 4 insults, total of 152 ml saline) and rewet except for Sample No 15. The following results were obtained.

| | FIUP 1st | FIUP 2nd | FIUP 3rd | FIUP 4th | |
|---|---|---|---|---|---|
| Sample | intake | intake | intake | intake | Rewet |
| No. | (sec) | (sec) | (sec) | (sec) | (g) |
| 13 | 16.48 | 28.88 | 45.9 | 75.98 | 0.75 |
| 14 | 15.24 | 28.52 | 46.32 | 61.34 | 0.39 |
| 16 | 18.56 | 41.1 | 71.74 | 97.68 | 0.37 |
| 17 | 18.18 | 40.04 | 67.7 | 92.8 | 0.33 |
| 18 | 17.2 | 31.52 | 52.8 | 81.8 | 0.52 |
| 19 | 22.7 | 51.3 | 81.4 | 136 | 0.54 |
| 20 | 20.42 | 51.7 | 87.2 | 115 | 0.20 |
| 21 | 19.22 | 47.82 | 80 | 107.5 | 0.33 |
| 22 | 19.76 | 55.34 | 93.44 | 118.9 | 0.31 |

All of the substrates displayed excellent fluid control properties. As shown, substrates made according the present disclosure can display an FIUP intake of less than about 20 seconds, such as less than about 18 seconds for the first intake, can display an FIUP intake of less than about 60 seconds, such as less than about 50 seconds, such as less than about 40 seconds, such as less than about 30 seconds for the second intake, can display an FIUP intake of less than about 95 seconds, such as less than about 85 seconds, such as less than about 70 seconds, such as less than about 55 seconds for the third intake, can display an FIUP intake of less than about 140 seconds, such as less than about 120 seconds, such as less than about 100 seconds, such as less than about 85 seconds for the fourth intake, and can display a rewet of less than about 0.8 grams, such as less than about 0.7 grams, such as less than about 0.6 grams, such as less than about 0.5 grams, such as less than about 0.4 grams. Sample No. 14, for example, displayed an FIUP intake of 15 seconds for the first intake, 28 seconds for second intake, 46 seconds for the third intake, 61 seconds for the fourth intake, and a rewet of about 0.4 grams. Furthermore, it was observed that the samples displayed improved flexibility.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only and is not intended to limit the invention so further described in such appended claims.

1. A multi-layer substrate comprising:
a first layer containing a binder, wherein the binder comprises binder fibers, wherein the first layer contains binder fibers in an amount greater than about 20% by weight;
a second layer comprising a superabsorbent material; and
wherein the multi-layer substrate has a tensile strength in at least one direction of greater than about 333 gf/in and displays a 30 second AUL test result of greater than about 10 g/g.

2. (canceled)

3. A multi-layer substrate as defined in claim 1, wherein the multi-layer substrate has an area-normalized saturation capacity of greater than about 0.25 g/cm² and less than about 5 g/cm².

4. A multi-layer substrate as defined in claim 1, wherein the second layer comprises less than about 20% by weight binder fibers.

5. A multi-layer substrate as defined in claim 1, wherein the second layer comprises less than 5% by weight binder fibers based on a total weight of all fibers contained in the second layer.

6. A multi-layer substrate as defined in claim 1, wherein the multi-layer substrate has a tensile strength in at least one direction of greater than about 700 gf/in and less than about 10,000 gf/in.

7. A multi-layer substrate as defined in claim 1, wherein the multi-layer substrate displays a 30 second AUL test result of greater than about 12 g/g.

8. A multi-layer substrate as defined in claim 1, further comprising a third layer, the second layer being positioned between the first layer and the third layer.

9. A multi-layer substrate as defined in claim 8, wherein the first layer comprises a top exterior layer and the third layer comprises a bottom exterior layer, the first layer being configured to face a user when incorporated into an absorbent article.

10. A multi-layer substrate as defined in claim 1, wherein the second layer has a basis weight of greater than about 200 gsm and less than about 800 gsm.

11. A multi-layer substrate as defined in claim 1, wherein the second layer contains the superabsorbent material in an amount greater than about 10% by weight.

12. A multi-layer substrate as defined in claim 10, wherein the second layer further contains pulp fibers, synthetic polymer fibers, or mixtures thereof.

13. A multi-layer substrate as defined in claim 1, wherein the first layer contains binder fibers in an amount greater than about 30% by weight and in an amount less than about 100% by weight.

14. A multi-layer substrate as defined in claim 13, wherein the first layer contains first binder fibers and second binder fibers, the first binder fibers having a size of about 3 denier or greater and the second binder fibers having a size of less than 3 denier.

15. A multi-layer substrate as defined in claim 13, wherein the first layer further contains synthetic polymer fibers, pulp fibers, or mixtures thereof.

16. A multi-layer substrate as defined in claim 1, wherein the first layer has a basis weight of greater than about 35 gsm and less than about 100 gsm, and wherein the first layer contains from about 20% by weight to about 80% by weight binder fibers, the binder fibers being combined with synthetic polymer fibers.

17. A multi-layer substrate as defined in claim 1, wherein the first layer has a basis weight of less than about 35 gsm, and greater than about 10 gsm, and wherein the first layer contains from about 10% by weight to about 80% by weight binder fibers, the binder fibers being combined with pulp fibers and optionally with polymer synthetic fibers.

18. A multi-layer substrate as defined in claim 8 or 9, wherein the third layer has a basis weight of greater than about 5 gsm and less than about 50 gsm, and wherein the third layer contains binder fibers.

19. A multi-layer substrate as defined in claim 18, wherein the third layer further contains pulp fibers, such as crosslinked pulp fibers, the binder fibers contained in the third layer having a size of less than about 6 denier.

20. A multi-layer substrate as defined in claim 1, wherein the first layer contains less than 2% by weight, such as less than 1% by weight superabsorbent material or is free of superabsorbent material.

21. A multi-layer substrate as defined in claim 1, wherein the binder fibers comprise bicomponent fibers containing a core polymer component surrounded by a sheath polymer component or two polymer components in a side by side juxtaposition.

22. A multi-layer substrate as defined in claim 21, wherein the bicomponent fiber includes a first polymer component comprising a polyester polymer or a polypropylene polymer and a second polymer component comprising a polyethylene polymer.

23. An absorbent article comprising:
a fluid permeable liner;
an outer cover; and
an absorbent core positioned between the liner and the outer cover, the absorbent core comprising the multi-layer substrate as defined in any of the preceding claims.

24. An absorbent article as defined in claim 23, wherein the absorbent article comprises a diaper or a child pant.

25. An absorbent article as defined in claim 23, wherein the absorbent article comprises an adult incontinence product.
